(19) United States
(12) Patent Application Publication
(10) Pub. No.: US 2024/0197596 A1
(43) Pub. Date: Jun. 20, 2024

(54) COMPOUNDS USEFUL AS HAIR DYES

(71) Applicants: National University of Singapore, Singapore (SG); Regents of the University of Minnesota, Minneapolis, MN (US)

(72) Inventors: Gopalakrishnan Venkatesan, Singapore (SG); Giorgia Pastorin, Singapore (SG); Paul Bigliardi, Minneapolis, MN (US)

(73) Assignees: National University of Singapore, Singapore (SG); Regents of the University of Minnesota, Minneapolis, MN (US)

(21) Appl. No.: 18/552,314

(22) PCT Filed: Mar. 23, 2022
(86) PCT No.: PCT/SG2022/050156

§ 371 Date: Sep. 25, 2023

Related U.S. Application Data
(60) Provisional application No. 63/165,429, filed on Mar. 24, 2021.

(51) Int. Cl. A61K 8/41; A61K 8/49; A61Q 5/10; C07C 217/84; C07C 225/22; C07C 255/16; C07D 303/23; C09B 57/00

(52) U.S. Cl. CPC A61K 8/411; A61K 8/4973; A61Q 5/10; C07C 217/84; C07C 225/22; C07C 255/16; C07D 303/23; C09B 57/00

(57) ABSTRACT

Disclosed herein is a compound of formula I:

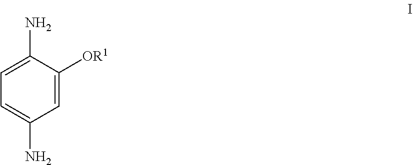

wherein the substituents are defined herein.

### FIELD OF INVENTION

The current invention relates to compounds and compositions containing said compounds that may act to provide a colouring to the skin or hair of a subject. The compounds disclosed herein may be more soluble in water, and/or less prone to causing an allergic or other immune response in a subject and are also oxidisable to a black colour and/or an intense dark colour.

### BACKGROUND

The listing or discussion of a prior-published document in this specification should not necessarily be taken as an acknowledgement that the document is part of the state of the art or is common general knowledge.

Hair and temporary skin colourings are among the most widely used cosmetic treatments today. Many of the dyes used in these treatments contain unstable di-/tri-functional aromatic amines that undergo oxidative polymerization to provide the desired pigmentation. However, the main component in dark dyes, para-phenylenediamine (PPD) or oxidative and enzymatic alterations of this diamine, can be irritants and very potent contact allergens. These potentially life-threatening allergies can appear as localized or generalized contact allergies with sometimes severe blister formations, itch and facial swellings and a potential to develop systemic reactions such as lymphadenopathy, asthma or methemoglobinemia, as well as fevers.

Para-phenylenediamine was nominated in 2006 as “allergene of the year”. It is a common amine-containing compound used particularly in hair dyes and non-permanent tattoos. PPD can penetrate easily through skin, where it can damage cells of the viable epidermis and interact with immune cells in the skin. This is also true for PPD derivatives produced by metabolic processes in the skin or by non-ionizing radiation (ultraviolet/visible/infrared light). In addition, the interaction of PPD and its derivatives with keratin proteins in the stratum corneum can form a depot, prolonging the release of PPD deeper into the skin, and making allergic reactions, particularly to PPD, a dangerous and long-lasting condition.

The experiments using 1% PPD solution from a standardized hair dresser patch test series (Chemotechnique) showed that when the PPD is contained within paraffin (as supplied), it is protected against oxidation. This is true even after 2 days of patch test as the test field remains colourless. The mass spectrometry measurements have shown that even after several days in air the PPD patch test formulation in paraffin does not show any Bandrowski base or other oxidation/metabolic derivatives in this formulation—only the parent compound PPD is present. However, sensitized patients develop a clear localised contact dermatitis in response to this PPD formulation. This suggests that PPD itself is the major allergen, either by haptenation or on its own (maybe through Toll-like receptor activation). Therefore, application of coloured polymers that cannot cross the skin barrier and which do not contain any PPD may potentially avoid the serious toxic and allergenic effects of hair dyes and temporary tattoos on skin and immune cells.

Commercially, a less sensitizing PPD derivative, ME-PPD was launched in 2018 as a safer permanent hair dye product that can replace PPD. However, approximately 30% of individuals who are PPD-allergic still develop an allergic reaction to ME-PPD, so they are recommended to avoid ME-PPD products. Hence, there appears to be no optimal non-toxic alternative to PPD for PPD-allergic individuals currently. Thus there remains a need for hair and temporary skin colourings that avoid such allergic responses and may be suitable for use in subjects who show sensitivity to conventional colouring materials.

### SUMMARY OF INVENTION

Some or all of the problems identified above may be solved by the current invention. Aspects and embodiments of the invention will now be discussed by reference to the following numbered clauses.
1. A compound of formula I:

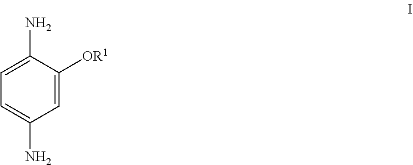

wherein:
R¹ represents C₁₋₁₀ alkyl substituted by one or more substituents selected from:
   halo, OR², CN, ═O, ═CH₂, NHR², and C₁₋₃ alkyl, which latter group is unsubstituted or substituted by halo, CN, OR³; and
   epoxy, provided that an epoxy substituent is only formed if R¹ is C₂₋₁₀ alkyl and two adjacent carbon atoms of R¹ together with an oxygen atom form the epoxy substituent,
R² represents H or C₁₋₅ alkyl, which is unsubstituted or substituted by one or more substituents selected from halo, OR⁴, and CN; and
R³ and R⁴ each independently represent H or C₁₋₃ alkyl, or a physiologically acceptable salt or solvate thereof.
2. The compound according to Clause 1, wherein:
R¹ represents C₁₋₁₀ alkyl substituted by one or more substituents selected from:
   halo, OR², CN, and C₁₋₃ alkyl, which latter group is unsubstituted or substituted by halo, CN, OR³; and
   epoxy, provided that an epoxy substituent is only formed if R¹ is C₂₋₁₀ alkyl and two adjacent carbon atoms of R¹ together with an oxygen atom form the epoxy substituent,
R² represents H or C₁₋₅ alkyl, which is unsubstituted or substituted by one or more substituents selected from halo, OR⁴, and CN; and
R³ and R⁴ each independently represent H or C₁₋₃ alkyl.
3. The compound according to Clause 1 or 2, wherein:
R¹ represents C₁₋₁₀ alkyl substituted by one or more substituents selected from:
   halo, OR², CN, and C₁₋₃ alkyl, which latter group is unsubstituted or substituted by halo, CN, OR³; and
   epoxy, provided that an epoxy substituent is only formed if R¹ is C₂₋₁₀ alkyl and two adjacent carbon atoms of R¹ together with an oxygen atom form the epoxy substituent,
R² represents C₁₋₅ alkyl, which is unsubstituted or substituted by one or more substituents selected from halo, OR⁴, and CN; and
R³ and R⁴ each independently represent C₁₋₃ alkyl.
4. The compound according to any one of the preceding clauses, wherein in the compound of formula I, R¹ represents C₁₋₆ alkyl, which is substituted with one or more substituents selected from OR², CN and epoxy, provided that an epoxy substituent is only formed if R¹ is C₂₋₆ alkyl and two adjacent carbon atoms of R¹ together with an oxygen atom form the epoxy substituent.
5. The compound according to Clause 4, wherein in the compound of formula I, R¹ represents C₁₋₃ alkyl, which is substituted with one or more substituents selected from OR², CN and epoxy, provided that an epoxy substituent is only formed if R¹ is C₂₋₃ alkyl and two adjacent carbon atoms of R¹ together with an oxygen atom form the epoxy substituent.
6. The compound according to Clause 5, wherein in the compound of formula I, R¹ represents C₁₋₃ alkyl, which is substituted with one or more substituents selected from CN and epoxy, provided that an epoxy substituent is only formed if R¹ is C₂₋₃ alkyl and two adjacent carbon atoms of R¹ together with an oxygen atom form the epoxy substituent.
7. The compound according to any one of the preceding clauses, wherein when present in the compound of formula I, R² represents C₁₋₃ alkyl, which is unsubstituted or substituted by one or more OR⁴ substituents.
8. The compound according to Clause 7, wherein when present in the compound of formula I, R² represents C₁₋₂ alkyl, which is unsubstituted or substituted by one or more OR⁴ substituents.
9. The compound according to any one of the preceding clauses, wherein when present in the compound of formula I, R³ and R⁴ each independently represent methyl.
10. The compound according to Clause 1 or 2, wherein:
R¹ represents C₁₋₄ alkyl substituted by one substituents selected from:
   OR², ═O, ═CH₂, and NHR²;
R² represents H or C₁₋₂ alkyl which is unsubstituted or substituted by an OR⁴ group;
R⁴ represents H or methyl.
11. The compound according to any one of the preceding clauses, wherein:
(a) R¹ is not substituted by C₁₋₃ alkyl, which C₁₋₃ alkyl is unsubstituted or substituted by halo, CN, OR³; and/or
(b) R¹ represents C₁₋₆ alkyl, which is substituted with one or more substituents selected from halo, OR², CN, ═O, ═CH₂, NHR², and epoxy, provided that an epoxy substituent is only formed if R¹ is C₂₋₆ alkyl and two adjacent carbon atoms of R¹ together with an oxygen atom form the epoxy substituent; and/or
(c) R¹ represents C₁₋₃ alkyl, which is substituted with one or more substituents selected from halo, OR², CN, ═O, ═CH₂, NHR², and epoxy, provided that an epoxy substituent is only formed if R¹ is C₂₋₃ alkyl and two adjacent carbon atoms of R¹ together with an oxygen atom form the epoxy substituent; and/or
(d) when present, R² represents H or C₁₋₃ (e.g. C₁₋₂) alkyl, which is unsubstituted or substituted by one or more OR⁴ substituents; and/or
(e) when present, R² represents H; and/or
(f) when present, R⁴ represents H or C₁₋₂ alkyl; and/or
(g) when present, R⁴ represents H; and/or
(h) when present, R³ is not H.
12. The compound according to Clause 1, wherein the compound of formula I is selected from the list:
(i) 2-(2,2-dimethoxyethoxy)benzene-1,4-diamine;
(ii) 2-(methoxymethoxy)benzene-1,4-diamine;
(iii) 2-(ethoxymethoxy)benzene-1,4-diamine;
(iv) 2-(oxiran-2-ylmethoxy)benzene-1,4-diamine;
(v) 2-[2-(2-methoxyethoxy)ethoxy]benzene-1,4-diamine;
(vi) 2-(2,5-diaminophenoxy)acetonitrile;
(vii) 2-(2-methoxyethoxy)benzene-1,4-diamine;
(viii) 2,5-diaminophenoxy)methoxy)methanol;
(ix) 2-(2,5-diaminophenoxy)ethan-1-ol;
(x) 1-(2,5-diaminophenoxy)propan-2-ol;
(xi) 1-(2,5-diaminophenoxy)butan-2-one;
(xii) 2-(prop-1-en-2-yloxy)benzene-1,4-diamine; and
(xiii) 2-(2-(methylamino)ethoxy)benzene-1,4-diamine, or
   a physiologically acceptable salt or solvate thereof.
   13. The compound according to Clause 1, wherein the compound of formula I is selected from the list:
(i) 2-(2,2-dimethoxyethoxy)benzene-1,4-diamine;
(ii) 2-(methoxymethoxy)benzene-1,4-diamine;
(iii) 2-(ethoxymethoxy)benzene-1,4-diamine;
(iv) 2-(oxiran-2-ylmethoxy)benzene-1,4-diamine;
(v) 2-[2-(2-methoxyethoxy)ethoxy]benzene-1,4-diamine;
(vi) 2-(2,5-diaminophenoxy)acetonitrile; and
(vii) 2-(2-methoxyethoxy)benzene-1,4-diamine, or
   a physiologically acceptable salt or solvate thereof.
   14. The compound according to Clause 13, wherein the compound of formula I is selected from the list:
(a) 2-(oxiran-2-ylmethoxy)benzene-1,4-diamine;
(b) 2-[2-(2-methoxyethoxy)ethoxy]benzene-1,4-diamine;
(c) 2-(2,5-diaminophenoxy)acetonitrile; and
(d) 2-(2-methoxyethoxy)benzene-1,4-diamine, or
   a physiologically acceptable salt or solvate thereof.
   15. The compound according to Clause 14, wherein the compound of formula I is selected from the list:
(a) 2-(oxiran-2-ylmethoxy)benzene-1,4-diamine; and
(b) 2-(2,5-diaminophenoxy)acetonitrile;
   a physiologically acceptable salt or solvate thereof.
   16. The compound according to Clause 12, wherein the compound of formula I is selected from the list:
(viii) 2,5-diaminophenoxy)methoxy)methanol;
(ix) 2-(2,5-diaminophenoxy)ethan-1-ol;
(x) 1-(2,5-diaminophenoxy)propan-2-ol;
(xi) 1-(2,5-diaminophenoxy)butan-2-one;
(xii) 2-(prop-1-en-2-yloxy)benzene-1,4-diamine; and
(xiii) 2-(2-(methylamino)ethoxy)benzene-1,4-diamine, or
   a physiologically acceptable salt or solvate thereof.
   17. The compound according to Clause 16, wherein the compound of formula I is selected from the list:
(viii) 2,5-diaminophenoxy)methoxy)methanol;
(ix) 2-(2,5-diaminophenoxy)ethan-1-ol; and
(x) 1-(2,5-diaminophenoxy)propan-2-ol, or
   a physiologically acceptable salt or solvate thereof.
   18. A method of dyeing hair or of applying a temporary tattoo, which method comprises applying a composition comprising a compound of formula I or a physiologically acceptable salt or solvate as described in any one of Clauses 1 to 17, or an oxidised derivative thereof.
   19. A composition for dyeing hair or tattooing skin, comprising:
      a compound of formula I as defined in any one of Clauses 1 to 17; and
      water.
   20. The composition of Clause 19, further comprising a coupling agent.
   21. A kit of parts comprising:
      (i) a composition according to Clause 19 or Clause 20; and
      (ii) a developing composition comprising an oxidising agent.

### DRAWINGS

Certain embodiments of the present disclosure are described more fully hereinafter with reference to the accompanying drawings.

FIG. 1. Cell viability profiles of N1-N13 on HaCaT skin cells. The graphs are represented by % cell viability on y-axis (linear scale) and concentration on x-axis (log scale).

FIG. 2. Mean peptide depletion percentage (Cysteine 1:10/Lysine 1:50) of N1-N13 after 24 h of incubation. Data represents mean±s.d of triplicate replicates, ns; non-significant, **; p value of <0.01, ***; p-value of <0.001. # indicates data from J Hazard Mat., 2021, 402, 123712.

FIGS. 3i and 3ii. Mean cumulative amount profiles of N1-N13 derivatives obtained from porcine skin permeation study performed in Franz diffusion cells. Cumulative amount profiles of the synthesized derivatives A. N1, B. N2, C.N3, D.N4, E.N5, F.N6, G.N7, H.N8, L.N9, J.N10, K.N11, L.N12, M.N13. Skin Data are means±s.d. of 6 replicates.

FIG. 4. Skin permeation of N1-N13 compared to PPD, ME-PPD and PTD, expressed as percentage of applied compound dose. A. Parent compound recovery in donor compartment, B) Parent compound permeation, C. Parent compound recovery in Skin. Data are means±s.d. of 6 replicates, ** p<0.01. # indicates data from J Hazard Mat., 2021, 402, 123712.

FIGS. 5i and 5ii. Extracted Ion chromatogram (XIC) of N1-N13 from skin permeation study. (A) XIC of N1, (B) XIC of N2, (C) XIC of N3, (D) XIC of N4, (E) XIC of N5, (F) XIC of N6, (G) XIC of N7, (H) XIC of N8, (I) XIC of N9, (J) XIC of N10, (K) XIC of N11, (L) XIC of N12, (M) XIC of N13.

FIG. 6. Elicitation of IL-8 and IL-1α by novel hair dyes and standards. Data represent the mean±SD of 6 independent experiments. Data are means±S.D of 6 replicates, ** p<0.01, *** p<0.001, differences compared with the negative control. Data for PPD, MEPPD and PTD from J Hazard Mat., 2021, 402, 123712.

FIGS. 7i to 7iii. CD54 and CD86 expression levels of N1-N13 on THP-1 cells were seeded at 1×10⁶ cells/mL, treated with each chemical at eight doses on CV75. (A) DNCB (positive control), (B) SLS (negative control), (C) PPD, (D) ME-PPD, (E) PTD, (F) N1, (G) N2, (H) N3, (I) N4, (J) N5, (K) N6, (L) N7, (M) N8, (M) N9, (O) N10, (P) N11, (Q) N12 and (R) N13, for 24 h and stained with the antibodies. CD54 and CD86 expressions were measured using flow cytometry. The graph denotes % RFI and % cell viability values on primary and secondary y-axes and compound concentration on the x-axis. The dashed and dotted lines represent OECD cutoff ranges of % RFI 200 (CD54) or % RFI 150 (CD86), respectively. % RFI values are mean±SEM values of three experiments.

FIGS. 8i to 8iv. Hair dye composition and eventual colours after N1-N13 derivatives were applied onto bleached hair samples.

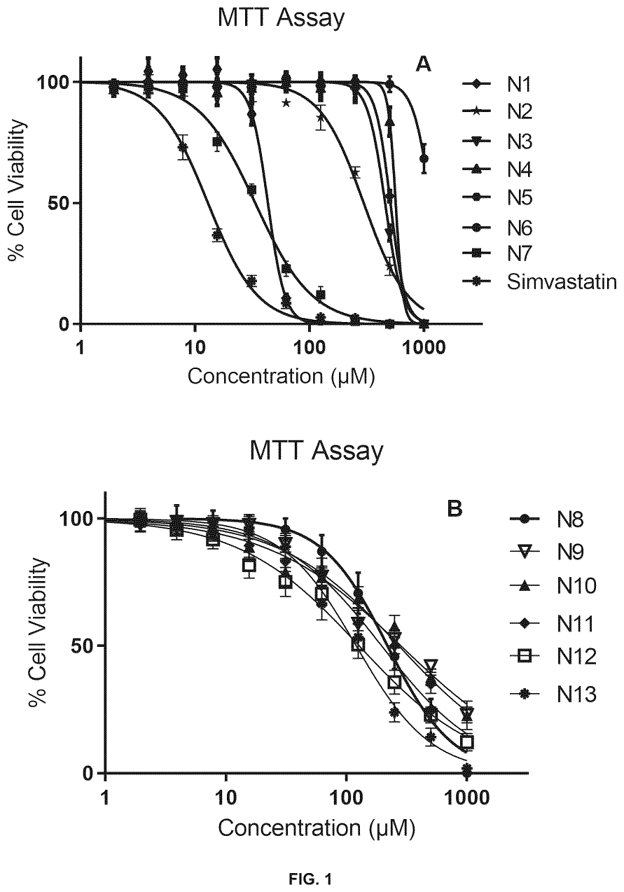

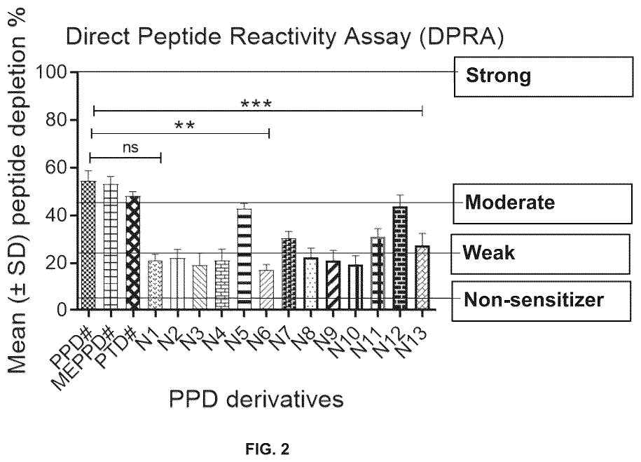

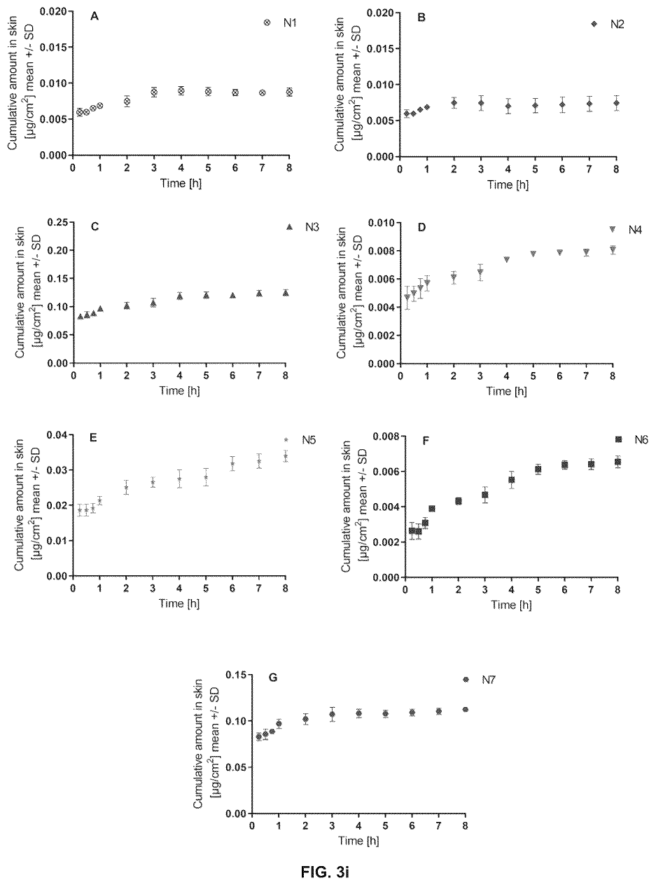

### DESCRIPTION

The current invention overcomes the problems identified above through the use of analogues of PPD that retain the dyeing effect (with or without oxidation), but avoid the sensitizing issues associated with PPD and/or its oxidation products. That is, the compounds disclosed hereinbelow may not provoke an allegoric or other immune reaction from a subject when used for the purposes of dyeing hair or colouring the skin.

Thus, according to a first aspect of the invention, there is provided a compound of formula I:

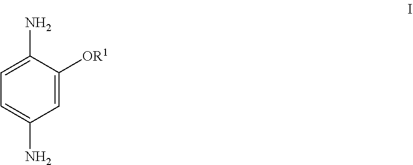

wherein:
R¹ represents C₁₋₁₀ alkyl substituted by one or more substituents selected from:
   halo, OR², CN, ═O, ═CH₂, NHR², and C₁₋₃ alkyl, which latter group is unsubstituted or substituted by halo, CN, OR³; and
   epoxy, provided that an epoxy substituent is only formed if R¹ is C₂₋₁₀ alkyl and two adjacent carbon atoms of R¹ together with an oxygen atom form the epoxy substituent,
R² represents H or C₁₋₅ alkyl, which is unsubstituted or substituted by one or more substituents selected from halo, OR⁴, and CN; and
R³ and R⁴ each independently represent H or C₁₋₃ alkyl, or a physiologically acceptable salt or solvate thereof.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

References herein (in any aspect or embodiment of the invention) to compounds of formula I includes references to such compounds per se, to tautomers of such compounds, as well as to physiologically acceptable salts or solvates, or oxidised derivatives of such compounds. Physiologically acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of physiologically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula I are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

“Oxidised derivatives” of compounds of formula I as defined herein are compounds that may be obtained from the compounds of formula I that are exposed to an oxidising agent, such as hydrogen peroxide. It will be appreciated that the resulting oxidised compounds may be analogous to the oxidation products of PPD.

Compounds of formula I, as well as physiologically acceptable salts, solvates and oxidised derivatives of such compounds are, for the sake of brevity, hereinafter referred to together as the “compounds of formula I”.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention.

Compounds of formula I may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

For the avoidance of doubt, in the context of the present invention, the terms “dyeing” and “tattooing” refers to the application of a cosmetic treatment to a body part of a subject, such as the skin (in the case of tattooing) or the hair (in the case of dyeing).

As used herein the terms “subject” is well-recognized in the art, and, is used herein to refer to a mammal, including dog, cat, rat, mouse, monkey, cow, horse, goat, sheep, pig, camel, and, most preferably, a human. The term does not denote a particular age or sex. Thus, adult and newborn (in some cases) subjects, whether male or female, are intended to be covered.

It will be appreciated that an effective amount of the compound of formula I, may be used to effect the dyeing and/or temporary tattooing of a subject. The term “effective amount” refers to an amount of a compound, which confers the desired colouring effect on the treated subject (e.g. sufficient to dye hair or temporarily tattoo skin). The effect may be objective (i.e. measurable by some test or marker) or subjective (i.e. the subject can see the difference).

The term “halo”, when used herein, includes references to fluoro, chloro, bromo and iodo.

Unless otherwise stated, the term “alkyl” refers to an unbranched or branched, cyclic, saturated or unsaturated (so forming, for example, an alkenyl or alkynyl) hydrocarbyl radical, which may be substituted or unsubstituted (with, for example, one or more halo atoms). Where the term “alkyl” refers to an acyclic group, it is preferably C₁₋₁₀ alkyl and, more preferably, C₁₋₅ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₁₂ cycloalkyl and, more preferably, C₅₋₁₀ (e.g. C₅₋₇) cycloalkyl. More particularly, the term alkyl will refer to an unbranched or branched, saturated hydrocarbyl radical that may be substituted or unsubstituted (with, for example, one or more halo atoms).

Further aspects and embodiments of the invention are provided in the following non-limiting examples.

In embodiments of the invention that may be mentioned herein,
R¹ may represent C₁₋₁₀ alkyl substituted by one or more substituents selected from:
   halo, OR², CN, and C₁₋₃ alkyl, which latter group is unsubstituted or substituted by halo, CN, OR³; and
   epoxy, provided that an epoxy substituent is only formed if R¹ is C₂₋₁₀ alkyl and two adjacent carbon atoms of R¹ together with an oxygen atom form the epoxy substituent,
R² may represent H or C₁₋₅ alkyl, which is unsubstituted or substituted by one or more substituents selected from halo, OR⁴, and CN; and
R³ and R⁴ may each independently represent H or C₁₋₃ alkyl.

In some embodiments of the invention that may be mentioned herein, R³ may represent C₁₋₃ alkyl.

In further embodiments of the invention that may be mentioned herein,
R¹ may represent C₁₋₁₀ alkyl substituted by one or more substituents selected from:
   halo, OR², CN, and C₁₋₃ alkyl, which latter group is unsubstituted or substituted by halo, CN, OR³; and
   epoxy, provided that an epoxy substituent is only formed if R¹ is C₂₋₁₀ alkyl and two adjacent carbon atoms of R¹ together with an oxygen atom form the epoxy substituent,
R² may represent C₁₋₅ alkyl, which is unsubstituted or substituted by one or more substituents selected from halo, OR⁴, and CN; and
R³ and R⁴ may each independently represent C₁₋₃ alkyl.

In embodiments of formula I that may be mentioned herein one or more of the following may apply:
(a) R¹ represents C₁₋₆ alkyl, which is substituted with one or more substituents selected from OR², CN and epoxy, provided that an epoxy substituent is only formed if R¹ is C₂₋₆ alkyl and two adjacent carbon atoms of R¹ together with an oxygen atom form the epoxy substituent,
   optionally R¹ may represent C₁₋₃ alkyl, which is substituted with one or more substituents selected from CN and epoxy, provided that an epoxy substituent is only formed if R¹ is C₂₋₃ alkyl and two adjacent carbon atoms of R¹ together with an oxygen atom form the epoxy substituent;
(b) when present in the compound of formula I, R² represents C₁₋₂ alkyl, which is unsubstituted or substituted by one or more OR⁴ substituents;
(c) when present in the compound of formula I, R³ and R⁴ each independently represent methyl.

In further embodiments of formula I one or more of the following may apply:
(i) R¹ represents C₁₋₃ alkyl, which is substituted with one or more substituents selected from OR², CN and epoxy, provided that an epoxy substituent is only formed if R¹ is C₂₋₃ alkyl and two adjacent carbon atoms of R¹ together with an oxygen atom form the epoxy substituent;
(ii) when present in the compound of formula I, R² represents C₁₋₃ alkyl, which is unsubstituted or substituted by one or more OR⁴ substituents.

It will be appreciated that one or more of (a) to (c) and (i) to (ii) may be selected. For example, R¹ may be selected from the definition provided in (ii) above, R² may be as defined in keeping with the broadest generic definition of the substituent, while R³ and R⁴ may be defined by (c) above.

In further embodiments of the invention that may be mentioned herein:
R¹ may represent C₁₋₄ alkyl substituted by one substituents selected from:
   OR², ═O, ═CH₂, and NHR²;
R² may represent H or C₁₋₂ alkyl which is unsubstituted or substituted by an OR⁴ group; and
R⁴ may represent H or methyl.

In yet further embodiments of the invention that may be mentioned herein, one or more of (a) to (h) may apply:
(a) R¹ is not substituted by C₁₋₃ alkyl, which C₁₋₃ alkyl is unsubstituted or substituted by halo, CN, OR³; and/or
(b) R¹ represents C₁₋₆ alkyl, which is substituted with one or more substituents selected from halo, OR², CN, ═O, ═CH₂, NHR², and epoxy, provided that an epoxy substituent is only formed if R¹ is C₂₋₆ alkyl and two adjacent carbon atoms of R¹ together with an oxygen atom form the epoxy substituent; and/or
(c) R¹ represents C₁₋₃ alkyl, which is substituted with one or more substituents selected from halo, OR², CN, ═O, ═CH₂, NHR², and epoxy, provided that an epoxy substituent is only formed if R¹ is C₂₋₃ alkyl and two adjacent carbon atoms of R¹ together with an oxygen atom form the epoxy substituent; and/or
(d) when present, R² represents H or C₁₋₃ (e.g. C₁₋₂) alkyl, which is unsubstituted or substituted by one or more OR⁴ substituents; and/or
(e) when present, R² represents H; and/or
(f) when present, R⁴ represents H or C₁₋₂ alkyl; and/or
(g) when present, R⁴ represents H; and/or
(h) when present, R³ is not H.

Other compounds of formula I that may be mentioned per se include compounds of the examples described hereinafter. Thus, embodiments of the invention that may be mentioned include those in which the compound of formula I is a compound selected from the list:
(i) 2-(2,2-dimethoxyethoxy)benzene-1,4-diamine [N1];
(ii) 2-(methoxymethoxy)benzene-1,4-diamine [N2];
(iii) 2-(ethoxymethoxy)benzene-1,4-diamine [N3];
(iv) 2-(oxiran-2-ylmethoxy)benzene-1,4-diamine [N4];
(v) 2-[2-(2-methoxyethoxy)ethoxy]benzene-1,4-diamine [N5];
(vi) 2-(2,5-diaminophenoxy)acetonitrile [N6];
(vii) 2-(2-methoxyethoxy)benzene-1,4-diamine [N7];
(viii) 2,5-diaminophenoxy)methoxy)methanol [N8];
(ix) 2-(2,5-diaminophenoxy)ethan-1-ol [N9];
(x) 1-(2,5-diaminophenoxy)propan-2-ol [N10];
(xi) 1-(2,5-diaminophenoxy)butan-2-one [N11];
(xii) 2-(prop-1-en-2-yloxy)benzene-1,4-diamine [N12]; and
(xiii) 2-(2-(methylamino)ethoxy)benzene-1,4-diamine [N13], or
   a physiologically acceptable salt or solvate thereof.

In more particular embodiments of the invention that may be mentioned herein, include those in which the compound of formula I is a compound selected from the list:
(i) 2-(2,2-dimethoxyethoxy)benzene-1,4-diamine [N1];
(ii) 2-(methoxymethoxy)benzene-1,4-diamine [N2];
(iii) 2-(ethoxymethoxy)benzene-1,4-diamine [N3];
(iv) 2-(oxiran-2-ylmethoxy)benzene-1,4-diamine [N4];
(v) 2-[2-(2-methoxyethoxy)ethoxy]benzene-1,4-diamine [N5];
(vi) 2-(2,5-diaminophenoxy)acetonitrile [N6]; and
(vii) 2-(2-methoxyethoxy)benzene-1,4-diamine [N7], or
   a physiologically acceptable salt or solvate thereof.

In yet more particular embodiments of the invention that may be mentioned herein, include those in which the compound of formula I is a compound selected from the list:
(a) 2-(oxiran-2-ylmethoxy)benzene-1,4-diamine [N4];
(b) 2-[2-(2-methoxyethoxy)ethoxy]benzene-1,4-diamine [N5];
(c) 2-(2,5-diaminophenoxy)acetonitrile [N6]; and
(d) 2-(2-methoxyethoxy)benzene-1,4-diamine [N7], or
   a physiologically acceptable salt or solvate thereof.

In yet more particular embodiments of the invention that may be mentioned herein, include those in which the compound of formula I is a compound selected from the list:
(a) 2-(oxiran-2-ylmethoxy)benzene-1,4-diamine [N4]; and
(b) 2-(2,5-diaminophenoxy)acetonitrile [N6];
   a physiologically acceptable salt or solvate thereof.

In yet more particular embodiments of the invention that may be mentioned herein, include those in which the compound of formula I is a compound selected from the list:
(viii) 2,5-diaminophenoxy)methoxy)methanol [N8];
(ix) 2-(2,5-diaminophenoxy)ethan-1-ol [N9];
(x) 1-(2,5-diaminophenoxy)propan-2-ol [N10];
(xi) 1-(2,5-diaminophenoxy)butan-2-one [N11];
(xii) 2-(prop-1-en-2-yloxy)benzene-1,4-diamine [N12]; and
(xiii) 2-(2-(methylamino)ethoxy)benzene-1,4-diamine [N13], or
   a physiologically acceptable salt or solvate thereof.

In yet more particular embodiments of the invention that may be mentioned herein, include those in which the compound of formula I is a compound selected from the list:
(viii) 2,5-diaminophenoxy)methoxy)methanol [N8];
(ix) 2-(2,5-diaminophenoxy)ethan-1-ol [N9]; and
(x) 1-(2,5-diaminophenoxy)propan-2-ol [N10], or
   a physiologically acceptable salt or solvate thereof.

It will be appreciated that the compounds of formula I may be useful in a composition for dyeing hair or (temporarily) tattooing the skin. Thus, in a further aspect of the invention, there is disclosed a composition for dyeing hair or tattooing skin, comprising a compound of formula I as defined above and water.

The term “temporary” in the context of tattoos in this invention is understood to mean a temporary colouring of the skin, which can be removed completely or nearly completely by washing (e.g. washing the tattoo with a soap) or by the natural shedding of the epidermis over a period of time.

In the context of the currently claimed invention, “dyeing hair” refers to the application of a formulation containing a compound of formula I to effect a permanent or semi-permanent colour change to the hair so dyed. This effect may be achieved without the presence of oxidative materials to blonde/bleach the hair and/or oxidise the compounds of formula I, though an oxidising material may be present in some embodiments described herein.

It will be appreciated that the compounds and compositions mentioned herein may be used for permanently dyeing hair. In which case, the effect is essentially permanent until the hair grows out or is dyed a different colour.

The composition may comprise from 0.0001 to 20 wt % of a compound of formula I, with the balance water. It will be appreciated that other components may form part of the composition, as discussed below, as such water is typically provided in an amount ranging from about 15% to about 99% by weight relative to the total weight of the composition. The pH range of the composition may be from about 1.0 to 14.0, though more typically, the pH range of the composition will be from about 3.0 to about 11.0. It will be appreciated that a combination of compounds of formula I are specifically contemplated herein. For the avoidance of doubt, reference to “compounds of formula I” also relates to physiologically acceptable salts or solvates, or an oxidised derivative thereof.

It will be appreciated that the compositions discussed herein may comprise additional components, which additional components may include, but are not limited to, coupling agents, surfactants, additional diluents/solvents, thickening agents, and alkalinising agents.

Any suitable coupling agent that may be used with PPD may be used herein, with the coupling agent forming from 0.0001 to 20 wt % of the entire composition. Suitable coupling agents may be selected from the group including, but not limited to, phenols, catechol, meta-aminophenols, meta-phenylenediamines, and the like, which may be unsubstituted, or substituted on the amino group or benzene ring with alkyl, hydroxyalkyl, alkylamino groups, and the like. Suitable couplers include 3,4-methylenedioxyphenol, 3,4-methylenedioxy-1-[(beta-hydroxyethyl)amino]benzene, 1-methoxy-2-amino-4-[(beta-hydroxyethyl)amino]-benzene, 1-hydroxy-3-(dimethylamino)benzene, 6-methyl-1-hydroxy-3[(beta-hydroxyethyl)-amino]benzene, 2,4-dichloro-1-hydroxy-3-aminobenzene, 1-hydroxy-3-(diethylamino)-benzene, 1-hydroxy-2-methyl-3-amninobenzene, 2-chloro-6-methyl-1-hydroxy-3-amino-benzene, 1,3-diaminobenzene, 6-methoxy-1,3-diaminobenzene, 6-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1,3-diaminobenzene, 6-methoxy-1-amino-3-[(beta-hydroxyethyl)amino]-benzene, 6-(beta-aminoethoxy)-1,3-diaminobenzene, 6-(beta-hydroxyethoxy)-1-amino-3-(methylamino)benzene, 6-carboxymethoxy-1,3-diaminobenzene, 6-ethoxy-1-bis(beta-hydroxyethyl)amino-3-aminobenzene, 6-hydroxyethyl-1,3-diaminobenzene, 1-hydroxy-2-isopropyl-5-methylbenzene, 1,3-dihydroxybenzene, 2-chloro-1,3-dihydroxybenzene, 2-methyl-1,3-dihydroxybenzene, 4-chloro-1,3-dihydroxybenzene, 5,6-dichloro-2-methyl-1,3-dihydroxybenzene, 1-hydroxy-3-amino-benzene, 1-hydroxy-3-(carbamoylmethylamino)benzene, 6-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxynaphthalene, 1,7-dihydroxynaphthalene, 1,5-dihydroxynaphthalene, 5-amino-2-methyl phenol, 4-hydroxyindole, 4-hydroxyindoline, 6-hydroxyindole, 6-hydroxyindoline, and mixtures thereof. Specific coupling agents that may be mentioned herein include resorcinol, 1-naphthol, 5-amino-o-cresol, 2-methylresorcinol, m-aminophenol, m-phenylenediamine, 1-phenyl-3-methyl-pyrazol-5-one, their salts, or mixtures

If the composition comprises a surfactant, the surfactant may be anionic, cationic, nonionic, zwitterionic or amphoteric. It will be appreciated that one or more surfactants may form part of the composition. When present, the surfactant(s) may form from 0.01 to 20 wt % of the composition.

Suitable nonionic surfactants that may be mentioned herein include, but are not limited to, alkyl polyglycosides, cetomacrogol 1000, cetostearyl alcohol, cetyl alcohol, cocamide DEA, cocamide MEA, decyl glucoside, decyl polyglucose, ethoxylates, glycerol monostearate, IGEPAL CA-630, isoceteth-20, lauryl glucoside, maltosides, monolaurin, mycosubtilin, nonidet P-40, nonoxynols, octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamers, polyethoxylated tallow amine, polyglycerol polyricinoleate, polysorbates, sorbitan, sorbitan monolaurate, sorbitan monostearate, sorbitan tristearate, stearyl alcohol, surfactin, Triton X-100, and Tween 80. Suitable cationic surfactants that may be mentioned herein include, but are not limited to, behentrimonium chloride, benzalkonium chloride, benzethonium chloride, benzododecinium bromide, bronidox, carbethopendecinium bromide, cetalkonium chloride, cetrimonium bromide, cetrimonium chloride, cetylpyridinium chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, octenidine dihydrochloride, olaflur, N-oleyl-1,3-propanediamine, pahutoxin, stearalkonium chloride, tetramethylammonium hydroxide, and thonzonium bromide.

Suitable zwitterionic surfactants that may be mentioned herein include, but are not limited to, betaines, N-alkyl-N,N-dimethylammonium glycinates, N-acylaminopropyl-N,N-dimethylammonium glycinates, and 2-alkyl-3-carboxymethyl-3-hydroxyethyl imidazolines.

Suitable zwitterionic surfactants that may be mentioned herein include, but are not limited to, N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids. Particularly preferred amphoteric surfactants are N-cocoalkyl aminopropionate, cocoacylaminoethyl aminopropionate, and C₁₂-C₁₈ acylsarcosine.

The compositions according to the present invention may include one or more solvents as additional diluent materials in addition to water. Generally, solvents suitable for use in the colouring compositions of the present invention are selected to be miscible with water and innocuous to the skin. Solvents suitable for use as additional diluents herein include C₁-C₂₀ mono- or polyhydric alcohols and their ethers, glycerine, with monohydric and dihydric alcohols and their ethers preferred. In these compounds, alcoholic residues containing 2 to 10 carbon atoms are preferred. Thus, a preferred group includes ethanol, isopropanol, n-propanol, butanol, propylene glycol, ethylene glycol monoethyl ether, and mixtures thereof. These additional diluents/solvents may be present in an amount of from about 0.5% to about 20% by weight of the total composition.

Depending on the selected application, the composition's viscosity may need to be adjusted viscosity. For example, this may be to meet consumer expectations or for functional reasons (e.g. to make the composition easier to handle for specific applications). This generally occurs through the use of one or more thickening agents. Any suitable thickening agent, such as organic and inorganic thickening agents may be used.

Suitable thickening agents include are anionic, synthetic polymers; cationic, synthetic polymers; naturally occurring thickeners, such as nonionic guar gums, scleroglucan gums or xanthan gums, gum arabic, gum ghatti, karaya gum, tragacanth gum, carrageenan gum, agar-agar, locust bean flour, pectins, alginates, starch fractions, and derivatives such as amylose, amylopectin, and dextrins, as well as cellulose derivatives (which are different from the celluloses of the invention) such as, for example, methylcellulose, carboxyalkylcelluloses, and hydroxyalkylcelluloses; nonionic, fully synthetic polymers such as polyvinyl alcohol or polyvinylpyrrolidinone; as well as inorganic thickeners, in particular phyllosilicates such as, for example, bentonite, in particular smectites, such as montmorillonite or hectorite. It will be appreciated that one or more of the above thickening agents may be used in the compositions described herein.

In compositions described herein, the thickening agents may be used in a total amount of from 0.1 to 4.5% by weight, such as from 0.15 to 3.5% by weight, such as from 0.2 to 2.0% by weight, based on the total weight of the composition.

The composition may have a pH range of from 7.0-10.0 (e.g. from 9.5 to 10.0). If the composition does not have a pH within the desired pH range for the application in question, then the pH may be adjusted by the addition of one or more alkalinising agents. Suitable alkalinising agents that can be used to adjust the desired pH value can be selected from the group formed by ammonia, alkanolamines, basic amino acids, and inorganic alkalinizing agents such as alkali (alkaline earth) metal hydroxides, alkali (alkaline earth) metal metasilicates, alkali (alkaline earth) metal phosphates, and alkali (alkaline earth) metal hydrogen phosphates. For example, the alkalinising agent may be Na₂CO₃.

If the pH is too alkaline, it will be appreciated that the composition may further comprise one or more acids to adjust the pH value. Suitable acids are, for example, organic acids such as alpha-hydroxycarboxylic acids or inorganic acids.

Further, the compositions described above may also include other active substances, auxiliary substances, and additives such as, for example, linear cationic polymers such as quaternized cellulose ethers, polysiloxanes with quaternary groups, dimethyldiallylammonium chloride polymers, acrylamide-dimethyldiallylammonium chloride copolymers, dimethylaminoethyl methacrylate-vinylpyrrolidinone copolymers quaternized with diethyl sulfate, vinylpyrrolidone-imidazolinium-methochloride copolymers, and quaternized polyvinyl alcohol; zwitterionic and amphoteric polymers; anionic polymers such as, for example, polyacrylic acids or crosslinked polyacrylic acids; structurants such as glucose, maleic acid, and lactic acid, hair-conditioning compounds such as phospholipids, for example, lecithin and kephalins; perfume oils, dimethyl isosorbide, and cyclodextrins; fiber-structure-improving active substances, particularly mono-, di-, and oligosaccharides such as, for example, glucose, galactose, fructose, fruit sugar, and lactose; dyes for colouring the agent; antidandruff agents such as piroctone olamine, zinc omadine, and climbazole; amino acids and oligopeptides; protein hydrolysates with an animal and/or vegetable base, and in the form of their fatty acid condensation products or optionally anionically or cationically modified derivatives; light stabilizers and UV blockers; active substances such as panthenol, pantothenic acid, pantolactone, allantoin, pyrrolidinone carboxylic acids and salts thereof, as well as bisabolol; polyphenols, particularly hydroxycinnamic acids, 6,7-dihydroxycoumarins, hydroxybenzoic acids, catechins, tannins, leukoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling and penetration agents such as glycerol, propylene glycol monoethyl ethers, carbonates, hydrogen carbonates, guanidines, ureas, and primary, secondary, and tertiary phosphates; opacifiers such as latex, styrene/PVP and styrene/acrylamide copolymers, and PEG-3 distearate; propellants such as propane-butane mixtures, N₂O, dimethyl ether, CO₂, and air.

The selection of these additional substances is made by the skilled artisan according to the desired properties of the composition in question. In regard to other facultative components and the employed amounts of said components, reference is made expressly to relevant handbooks known to the skilled artisan. The additional active and auxiliary substances are used in the agents of the invention preferably in each case in amounts of 0.0001 to 25% by weight, in particular of 0.0005 to 15% by weight, based on the total weight of the composition in question.

The compositions disclosed herein may be produced, for example, in the form of a lotion, a gel, a spray, an aerosol, or a pump foam. Depending on the application form, they are therefore preferably filled into a tube, a container, a bottle, a box, a pressurized container, or into a container with a pump spray applicator.

In certain embodiments, the composition disclosed above may contain oxidised derivatives of the compounds of formula I. In which case, there may be no need to provide a separate oxidizing agent. While compositions where the compounds of formula I are in an unoxidized form may be used as is—and provide at least temporary hair dyeing effects. It is also contemplated that an oxidizing agent may also be included into the composition (or applied separately) in order to blonde the hair and provide a more consistent colouration, which may be permanent. Thus, in a further aspect of the invention, there is provided a kit of parts comprising:
(i) a composition as described above; and
(ii) a developing composition comprising an oxidising agent.

It will be appreciated that the composition described above preferably contains unoxidised forms of the compounds of formula I, though oxidised forms of the compounds of formula I may also be used.

In general, the compositions comprising the compounds of formula I described above may be used in these kits of parts. As such, reference to the above description of these compositions is made here.

The developing composition is added to a composition comprising the compounds of formula I in order to provide a blonding/bleaching effect on hair and, possibly, to cause oxidation of the compounds of formula I (if in an non-oxidised form). The developing composition may use any suitable oxidizing agent that is safe for use on skin and hair. A suitable oxidising agent that may be mentioned herein is hydrogen peroxide. The oxidising agent may be provided in a suitable amount that falls within regulatory guidelines. As such, in accordance with the Cosmetic Directive of the European Union (Council Directive of 27 Jul. 1976 r. Annex III p. 12), the maximum permitted concentration in a ready-to-use hair dye is 12% (40 volumes) and 4 wt % in skin-care preparations. It will be appreciated that the kit of parts mentioned here is intended for use in hair dye compositions, as such the oxidising agent may be present in an amount of from 0.5 to 45 wt % in the developer composition, with the balance being water.

In further embodiments of the invention, the developer composition may further comprise a surfactant, a thickening agent, and an acidifying agent.

When present in the developer composition, one or more surfactants may be selected from those mentioned hereinbefore. The surfactant(s) may be present in an amount of from 0.01 to 20 wt % of the developer composition.

When present in the developer composition, one or more thickening agents may be selected from those mentioned hereinbefore. The thickening agent(s) may be present in an amount of from 0.01 to 20 wt % of the developer composition.

The developer composition may have a pH range of from 2.5-6.9. If the developer composition does not have a pH within the desired pH range for the application in question, then the pH may be adjusted by the addition of one or more acidifying agents. Suitable acidifying agents include, for example, organic acids such as alpha-hydroxycarboxylic acids or inorganic acids.

Further, the developer compositions may also include other active substances, auxiliary substances, and additives, as described hereinbefore.

As will be appreciated that compounds disclosed herein are useful for dyeing hair or providing a temporary tattoo on skin. Thus, in a further aspect of the invention, there is provided a method of dyeing hair or of applying a temporary tattoo, which method comprises applying a composition comprising a compound of formula I or a physiologically acceptable salt or solvate as described herein, or an oxidised derivative thereof.

When used in hair dyeing, the hair dyeing method may have three steps:
the first step requires contacting a subject's hair with the “composition” (upon optional mixing with a developer (e.g. a composition comprising an oxidizing agent));
allowing the mixture to remain in the hair for a period of about 30 seconds to about 60 minutes; and
then washing the hair extensively with water and surfactant-containing shampoos leaving the long-lasting hair colour change.

A temporary tattoo using compositions described herein, may be applied in a manner similar to henna tattoos to the skin of a subject.

The invention will be further described in connection with the following examples, which are set forth for the purposes of illustration only.

### EXAMPLES

### Materials and Methods

Unless otherwise mentioned, all the commercial materials were used without further purification. NMR spectra were recorded on a Bruker Ultrashield 300 MHz system, Bruker Ultrashield Plus 400 MHz system and Bruker Ultrashield 500 MHz system, at room temperature with DMSO-d6 as the solvent and TMS as the internal standard. ¹H and ¹³C chemical shifts are reported in ppm downfield of tetramethylsilane with reference to the residue solvent peak. Coupling constant (J) were reported in Hert (Hz) and signal couplings are reported using the following abbreviations: s, singlet; d, doublet; t, triplet; m, multiplet; p, pentet; q, quartet; br, broad resonance. High resolution mass spectra (HRMS) were obtained on a Finnigan/MAT 95XL-T spectrometer system equipped with electrospray ionization (ESI) and, or Atmospheric Pressure Chemical Ionization (APCI) mode.

Para-phenylenediamine (98.0%) and resorcinol (≥99.0%) were purchased from Sigma-Aldrich (Singapore). Schwarzkopf BlondMe Premium Lift 9+ bleaching powder and Schwarzkopf BlondMe 12%/40 vol developer solution, Silkpro VitAir series daily balance shampoo, and Silkpro VitAir series daily treatment Masque (hair conditioner) were purchased from Amazon and used without further purification. 30% hydrogen peroxide solution was purchased from Merck (Singapore).

HPLC grade acetonitrile (99.0%), methanol (99.0%) from Fisher Chemical (Fischer Scientific, Belgium), leucine enkephalin acetate salt hydrate (95.0%), deferoxamine Mesylate (92.5%), lysine-containing (Ac-RFAAKAA-COOH, 95.5%) and cysteine-containing (Ac-RFAACAA-COOH, 96.05%) heptapeptides were purchased from Peptide 2.0 Inc (Chantilly, VA) were purchased from Sigma-Aldrich (Singapore).

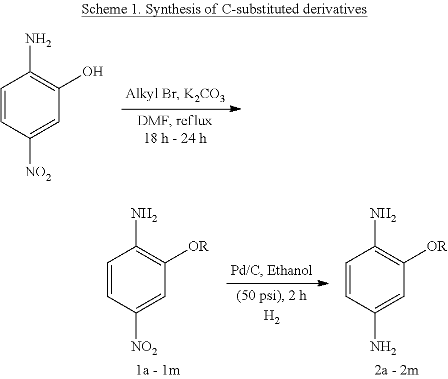

TABLE 1
Chemical Structure, Molecular Weight and cLogP values of N1-N13. *cLogP values
were calculated using ChemDraw ultra version 12.0.2.

| Code | Compound | Reagents (Alkyl Br) | —R | Molecular Weight | cLogP |
|---|---|---|---|---|---|
| N1 | a | 2-bromo-1,1- | —CH₂(CH)(OCH₃)₂ | 212.25 | −0.425 |
| | | dimethoxyethane | | | |
| N2 | b | Bromomethyl methyl | —CH₂OCH₃ | 168.20 | −0.485 |
| | | ether | | | |
| N3 | c | Chloromethyl ethyl ether | —CH₂OCH₂CH₃ | 182.22 | −0.096 |
| N4 | d | 1-bromo-2,3- epoxypropane | 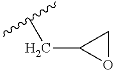 [SMILES: *CC1CO1] | 180.21 | −0.428 |
| N5 | e | 1-bromo-2-(2 methoxy | —CH₂CH₂OCH₂CH₂OCH₃ | 226.28 | −0.237 |
| | | ethoxy)ethane | | | |
| N6 | f | Bromoacetonitrile | —CH₂CN | 163.18 | −0.959 |
| N7 | g | 1-bromo-2- | —CH₂CH₂OCH₃ | 182.22 | −0.1022 |
| | | methoxyethane | | | |
| N8 | h | 2- | —CH₂—O—CH₂—OH | 108.14 | −0.312 |
| | | (bromomethoxy)methanol | | | |
| N9 | i | 2-bromoethanol | —CH₂—CH₂—OH | 168.20 | −0.865 |
| N10 | j | 1-bromopropane-2-ol | —CH₂(CH)OH—CH₃ | 182.22 | −0.556 |
| N11 | k | 1-bromo-2-butanone | —CH₂—CO—CH₂—CH₃ | 194.23 | −0.11 |
| N12 | l | 2-bromopropene | 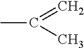 | 164.21 | 1.132 |
| N13 | m | 2-bromo-N- | —CH₂—CH₂—NH—CH₃ | 181.24 | −0.486 |
| | | methylethanamine | | | |

General Procedure 1: Synthesis of Nitro Intermediates 1a-1m

This relates to the synthesis of alkoxy derivatives by nucleophilic substitution of the bromo group of an alkylbromide.

2-amino-5-nitrophenol (0.0065M) was dissolved in 5 mL of DMF kept under an inert atmosphere and alkyl bromide (0.00715 M, 1.1 eq) and anhydrous K₂CO₃ (0.00715 M, 1.1 eq) were added to the solution. The resulting red mixture was refluxed for 18-24 hours, after which the resulting dark brown mixture was added dropwise to saturated NaHCO₃ solution with vigorous stirring. After 30 minutes, the mixture was extracted with DCM and the organic layer was washed 3 times with NaHCO₃ solution, 3 times with saturated LiCl solution and finally with brine solution. After drying over anhydrous Na₂SO₄, the solvent was removed under reduced pressure and the resulting mixture oil dried in vacuo. The oily mixture was then purified by gradient column chromatography with ethyl acetate and hexane mixture as eluent. The compound was eluted with a mixture of ethyl acetate and hexane, and solvent evaporation afforded the intermediate product (1α-1m) as a bright yellow solid.

### General Procedure 2: Synthesis of C-Substituted Derivatives 2α-2m

This relates to the reduction of nitro groups to amino groups.

Nitro derivatives (0.001 M; prepared from General Procedure 1) were dissolved in anhydrous ethanol (50 ml) and to which 10% palladium on carbon (0.00012 M, 0.12 eq) was added. The mixture was shaken under hydrogen gas with 50 psi using Parr reactor for 2 hours. The reaction mixture was filtered using celite pad twice and the ethanolic filtrate was evaporated to obtain a final product (2α-2m) as solid.

### Example 1: Synthesis of N1

### Synthesis of Compound 1a

Compound 1a was prepared as a yellow-orange solid following General Procedure 1 using 2-bromo-1,1-dimethoxyethane as the alkyl bromide. 2-(2,2-dimethoxyethoxy)-4-nitroaniline (1a): Yield: 2.0 g (86.0%). ¹H NMR (400 MHz, DMSO-d₆) δ 8.10 (dd, J=2.4 Hz, 8.8 Hz, 1H), 7.90 (d, J=2.4 Hz, 1H), 6.92 (d, J=8.8 Hz, 1H), 6.25 (s, 2H), 4.68 (t, J=6.9 Hz, 1H), 4.23 (d, J=4.8 Hz, 2H), 3.32 (s, 6H).

### Synthesis of Compound 2a (N1)

Compound 2a was prepared as a dark brown solid from compound 1a following General Procedure 2. 2-(2,2-dimethoxyethoxy)benzene-1,4-diamine (2a): Yield: 0.98 g (98.0%).

¹H NMR (400 MHz, DMSO-d₆) δ 10.05 (s, 2H), 7.75 (dd, J=2.4 Hz, 8.8 Hz, 1H), 7.64 (d, J=2.0 Hz, 1H), 6.69 (d, J=8.8 Hz, 1H), 4.75 (s, 2H), 4.56 (t, J=5.2 Hz, 1H), 4.08 (d, J=4.8 Hz, 2H), 3.37 (s, 6H). ¹³C NMR (400 MHz, DMSO-d₆) δ 146.1, 143.3, 135.6, 119.9, 111.2, 107.6, 101.7, 68.4, 53.8. HRMS (ESI): calcd for C₁₀H₁₆N₂O₃[M+H]⁺: 213.1155, obsd: 213.1165, mass err (ppm): 4.6. HPLC purity (254 nm); 99.249%, eluent: 90% ACN/NH4OAc, tR=21.688 min.

### Example 2: Synthesis of N2

### Synthesis of Compound 1b

Compound 1b was prepared as a yellow solid following General Procedure 1 using bromomethyl methyl ether as the alkyl bromide. 2-(methoxymethoxy)-4-nitroaniline (1b): Yield: 2.3 g (66.0%). ¹H NMR (400 MHz, DMSO-d₆) δ 8.08 (dd, J=2.4 Hz, 8.6 Hz, 1H), 7.84 (d, J=2.4 Hz, 1H), 6.98 (d, J=8.8 Hz, 1H), 6.36 (s, 2H), 5.98 (s, 2H), 3.40 (s, 3H).

### Synthesis of Compound 2b (N2)

Compound 2b was prepared as a black solid from compound 1b following General Procedure 2. 2-(methoxymethoxy)benzene-1,4-diamine (2b): Yield: 0.92 g (92.0%).

¹H NMR (400 MHz, DMSO-d₆) δ 10.29 (s, 2H), 7.72 (dd, J=2.4 Hz, 8.8 Hz, 1H), 7.46 (d, J=2.4 Hz, 1H), 6.48 (d, J=8.8 Hz, 1H), 5.47 (s, 2H), 4.69 (br, 2H), 3.35 (s, 3H). ¹³C NMR (400 MHz, DMSO-d₆) δ 143.7, 142.9, 138.6, 119.9, 112.3, 110.0, 93.8, 59.6. HRMS (ESI): calcd for C₃H₁₂N₂O₂ [M+H]⁺: 169.0859, obsd: 169.0858, mass err (ppm): 0.5. HPLC purity (254 nm); 99.500%, eluent: 90% ACN/NH4OAc, tR=16.002 min.

### Example 3: Synthesis of N₃

### Synthesis of Compound 1c

Compound 1c was prepared as a yellowish brown solid following General Procedure 1 using Chloromethyl ethyl ether as the alkyl bromide. 2-(ethoxymethoxy)-4-nitroaniline (1c): Yield: 2.4 g (76.0%).

¹H NMR (400 MHz, DMSO-d₆) δ 8.14 (dd, J=2.4 Hz, 8.8 Hz, 1H), 7.94 (d, J=2.4 Hz, 1H), 6.96 (d, J=8.8 Hz, 1H), 6.51 (s, 2H), 5.54 (s, 2H), 3.85 (q, J=6.8 Hz, 2H), 1.32 (t, J=7.4 Hz, 3H).

### Synthesis of Compound 2c (N₃)

Compound 2c was prepared as a black solid from compound 1c following General Procedure 2. 2-(ethoxymethoxy)benzene-1,4-diamine (2c): Yield: 0.94 g (94.0%). ¹H NMR (400 MHz, CDCl₃) δ 7.92 (d, J=2.4 Hz, 1H), 7.81 (dd, J=2.4 Hz, 8.8 Hz, 1H), 6.65 (d, J=8.8 Hz, 1H), 5.31 (s, 2H), 3.74 (q, J=6.8 Hz, 2H), 1.24 (t, J=7.2 Hz, 3H). ¹³C NMR (400 MHz, CDCl₃) δ 143.9, 143.1, 138.7, 120.1, 112.5, 110.2, 93.9, 65.0, 15.2. HRMS (ESI): calcd for C₉H₁₄N₂O₂ [M+H]⁺: 183.1492, obsd: 183.1494, mass err (ppm): −1.2. HPLC purity (254 nm); 99.172%, eluent: 90% ACN/NH4OAc, tR=27.659 min.

### Example 4: Synthesis of N₄

### Synthesis of Compound 1d

Compound 1d was prepared as a yellow solid following General Procedure 1 using 1-bromo-2,3-epoxypropane as the alkyl bromide. 4-nitro-2-(oxiran-2-ylmethoxy)aniline (1d): Yield: 3.2 g (68.0%). ¹H NMR (400 MHz, DMSO-d₆) δ 8.05 (dd, J=2.4 Hz, 8.8 Hz, 1H), 7.86 (d, J=2.4 Hz, 1H), 6.89 (d, J=8.8 Hz, 1H), 6.47 (s, 2H), 5.05 (d, J=2.4 Hz, 1H), 4.69 (t, J=6.9 Hz, 1H), 4.0-4.1 (m, 1H), 3.83-3.92 (m, 1H), 3.48-3.50 (m, 1H). 4.13-4.18 (m, 1H), 3.99-4.05 (m, 2H), 3.49-3.55 (m, 2H).

### Synthesis of Compound 2d (N₄)

Compound 2d was prepared as a beige solid from compound 1d following General Procedure 2. 2-(oxiran-2-ylmethoxy)benzene-1,4-diamine (2d): Yield: 0.96 g (96.0%): ¹H NMR (400 MHz, DMSO-d₆) δ 10.35 (s, 2H), 7.74 (dd, J=2.4 Hz, 8.8 Hz, 1H), 7.56 (d, J=2.4 Hz, 1H), 6.66 (d, J=8.8 Hz, 1H), 4.69 (s, 2H), 4.08-4.12 (m, 1H), 3.83-3.92 (m, 2H), 3.47-3.51 (m, 2H). ¹³C NMR (400 MHz, DMSO-d₆) δ 146.3, 140.0, 127.9, 115.8, 107.6, 101.9, 67.9, 62.0, 53.8. HRMS (ESI): calcd for C₉H₁₂N₂O₂ [M+H]⁺: 181.0886, obsd: 181.0880, mass err (ppm): −3.3. HPLC purity (254 nm); 97.2%, eluent: 90% ACN/NH4OAc, tR=22.467 min.

### Example 5: Synthesis of N₅

### Synthesis of Compound 1e

Compound 1e was prepared as a yellowish brown solid following General Procedure 1 using 1-bromo-2-(2-methoxyethoxy)ethane as the alkyl bromide. 2-(2-(2-methoxyethoxy)ethoxy)-4-nitroaniline (1e): Yield: 0.78 g (78.2%). ¹H NMR (400 MHz, DMSO-d₆) δ 7.94 (dd, J=2.4 Hz, 8.8 Hz, 1H), 7.82 (d, J=2.4 Hz, 1H), 6.95 (d, J=8.8 Hz, 1H), 6.31 (s, 2H), 4.18 (t, J=6.4 Hz, 2H), 3.78 (t, J=6.4 Hz, 2H), 3.61 (t, J=6.4 Hz, 2H), 3.46 (t, J=6.4 Hz, 2H), 3.21 (s, 3H).

### Synthesis of Compound 2e (N₅)

Compound 2e was prepared as a dark brown solid from compound 1e following General Procedure 2. 2-[2-(2-methoxyethoxy)ethoxy]benzene-1,4-diamine (2e): Yield: 0.92 g (92.0%). ¹H NMR (400 MHz, D₂O) δ 7.52 (d, J=8.4 Hz, 1H), 7.18 (d, J=2 Hz, 1H), 7.09 (dd, J=2 Hz, 8.4 Hz, 1H), 4.35 (t, J=4 Hz, 2H), 3.96 (t, J=4 Hz, 2H), 3.75-3.77 (m, 2H), 3.63-3.65 (m, 2H), 3.36 (s, 3H). ¹³C NMR (400 MHz, D₂O) δ 152.3, 132.3, 125.1, 119.7, 115.5, 108.0, 70.9, 69.5, 68.6, 68.2, 58.0. HRMS (ESI): calcd for C₁₁H₁₈N₂O₃ [M+H]⁺: 227.1390, obsd: 227.1392, mass err (ppm): −0.8. HPLC purity (254 nm); 99.063%, eluent: 90% ACN/NH4OAc, tR=24.725 min.

### Example 6: Synthesis of N₆

### Synthesis of Compound 1f

Compound if was prepared as an orange solid following General Procedure 1 using bromoacetonitrile as the alkyl bromide. 2-(2-amino-5-nitrophenoxy)acetonitrile (1f): Yield: 0.52 g (52.6%). ¹H NMR (400 MHz, DMSO-d₆) δ 7.90 (dd, J=8.6 Hz, 2.4 Hz, 1H), 7.74 (d, J=2.4 Hz, 1H), 7.03 (d, J=8.8 Hz, 1H), 6.34 (s, 2H), 4.71 (s, 2H).

### Synthesis of Compound 2f (N₆)

Compound 2f was prepared as a purple crystalline solid from compound if following General Procedure 2. 2-(2,5-diaminophenoxy)acetonitrile (2f): Yield: 0.94 g (94.0%): ¹H NMR (400 MHz, DMSO-d₆) δ 10.27 (s, 2H), 6.57 (d, J=8.0 Hz, 1H), 6.15-6.19 (m, 2H), 4.87 (s, 2H), 4.41 (s, 2H). ¹³C NMR (400 MHz, DMSO-d₆) δ 164.9, 143.9, 127.4, 127.0, 117.5, 116.8, 111.8, 67.1. HRMS (ESI): calcd for C₃H₉N₃O [M+H]⁺: 164.0580, obsd: 164.0579, mass err (ppm): 0.9. HPLC purity (254 nm); 98.988%, eluent: 90% ACN/NH4OAc, tR=13.843 min.

### Example 7: Synthesis of N₇

### Synthesis of Compound 1g

Compound 1g was prepared as a yellow solid following General Procedure 1 using 1-bromo-2-methoxyethane as the alkyl bromide. 2-(2-methoxyethoxy)-4-nitroaniline (1g): Yield: 0.88 g (88.0%). ¹H NMR (400 MHz, DMSO-d₆) δ 8.0 (dd, J=8.8 Hz, 2.6 Hz, 1H), 7.90 (d, J=2.4 Hz, 1H), 7.02 (d, J=8.8 Hz, 1H), 6.38 (s, 2H), 4.35 (t, J=6.8 Hz, 2H), 3.85 (t, J=6.8 Hz, 2H), 3.18 (s, 3H).

### Synthesis of Compound 2g (N₇)

Compound 2g was prepared as a beige solid from compound 1g following General Procedure 2. 2-(2-methoxyethoxy)benzene-1,4-diamine (2g): Yield: 0.98 g (98.0%). ¹H NMR (400 MHz, DMSO-d₆) δ 10.1 (s, 2H), 7.74 (dd, J=2 Hz, 8.8 Hz, 1H), 7.61 (d, J=2.4 Hz, 1H), 6.68 (d, J=8.8 Hz, 1H), 4.76 (s, 2H), 4.19 (t, J=4.4 Hz, 2H), 3.71 (t, J=4.8 Hz, 2H), 3.35 (s, 3H). ¹³C NMR (400 MHz, DMSO-d₆) δ 146.2, 143.6, 135.6, 119.8, 111.1, 107.1, 70.2, 68.1, 58.2. HRMS (ESI): calcd for C₉H₁₄N₂O₂ [M+H]⁺: 183.1128, obsd: 183.1127, mass err (ppm): 0.8. HPLC purity (254 nm); 98.348%, eluent: 90% ACN/NH4OAc, tR=27.102 min.

### Synthesis of Compound 1h

Compound 1h was prepared as a yellow-orange solid following General Procedure 1 using 2 (bromomethoxy) methanol as the alkyl bromide. (2-amino-5-nitrophenoxy) methoxy) methanol (1h): Yield: 0.8 g (84%). ¹H NMR (400 MHz, DMSO-d6) δ 7.74 (dd, J=8.8 Hz, 2.4 Hz, 1H), 7.54 (d, J=2.4 Hz, 1H), 6.76 (d, J=8.8 Hz, 1H), 6.34 (s, 2H), 4.08 (s, 1H), 5.98 (s, 2H), 5.65 (s, 2H).

### Synthesis of Compound 2h (N₈)

Compound 2h was prepared as a dark brown solid from compound 1h following General Procedure 2. ((2,5-diaminophenoxy)methoxy)methanol, (2h) Yield: 0.75 g (75%). ¹H NMR (400 MHz, DMSO-d6) δ 6.90 (d, J=8.0 Hz, 1H), 6.34 (d, J=2.0 Hz, 1H), 6.20 (dd, J=8.4 Hz, 2.0 Hz, 1H), 5.82 (s, 2H), 5.40 (s, 2H), 4.72 (br, 4H), 4.08 (s, 1H). ¹³C NMR (300 MHz, DMSO-d6) δ 148.0, 141.0, 127.0, 117.5, 109.0, 98.8, 88.2, 87.5.

### Synthesis of Compound 1i

Compound 1i was prepared as a yellow-orange solid following General Procedure 1 using 2-bromoethanol as the alkyl bromide. 2-(2-amino-5-nitrophenoxy)ethan-1-ol (ii): Yield: 1.2 g (78%). ¹H NMR (400 MHz, DMSO-d6) δ 7.80 (dd, J=8.8 Hz, 2.4 Hz, 1H), 7.64 (d, J=2.4 Hz, 1H), 6.74 (d, J=8.8 Hz, 1H), 6.32 (s, 2H), 4.86 (s, 1H), 4.30 (t, J=6.4 Hz, 2H), 3.60 (t, J=6.2 Hz, 2H).

### Synthesis of Compound 2i (N₉)

Compound 2i was prepared as a brown solid from compound 1i following General Procedure 2. 2-(2,5-diaminophenoxy)ethan-1-ol, (2i): Yield: 0.82 g (82%). ¹H NMR (400 MHz, DMSO-d6) δ 6.70 (d, J=7.8 Hz, 1H), 6.29 (d, J=2.2 Hz, 1H), 5.65 (dd, J=8.5 Hz, 2.0 Hz, 1H), 4.90 (s, 2H), 4.70 (br, 4H), 4.33 (t, J=6.8 Hz, 3H), 3.72 (t, J=7.0 Hz, 3H). ¹³C NMR (400 MHz, DMSO-d6) δ 146.0, 141.5, 128.2, 118.5, 109.2, 100.8, 90.0, 61.2.

### Synthesis of Compound 1j

Compound 1j was prepared as a orange solid following General Procedure 1 using 1-bromopropane-2-ol as the alkyl bromide. 1-(2-amino-5-nitrophenoxy)propan-2-ol (1j): Yield: 1.4 g (80%). ¹H NMR (400 MHz, DMSO-d6) δ 7.82 (dd, J=8.8 Hz, 2.4 Hz, 1H), 7.68 (d, J=2.4 Hz, 1H), 6.80 (d, J=8.8 Hz, 1H), 6.42 (s, 2H), 5.30 (s, 1H), 4.16-4.19 (m, 1H), 3.90-3.94 (m, 2H), 1.06-1.09 (m, 3H).

### Synthesis of Compound 2j (N₁₀)

Compound 2j was prepared from as a brown solid compound 1h following General Procedure 2. 1-(2,5-diaminophenoxy)propan-2-ol, (2j): Yield: 0.65 g (65%). ¹H NMR (400 MHz, DMSO-d6) δ 6.72 (d, J=8.8 Hz, 2.4 Hz, 1H), 6.32 (d, J=2.4 Hz, 1H), 5.76 (dd, J=8.8 Hz, 1H), 5.25 (s, 1H), 4.80 (br, 4H), 4.17-4.20 (m, 1H), 3.90-3.94 (m, 2H), 1.06-1.09 (m, 3H). ¹³C NMR (400 MHz, DMSO-d6) δ 146.0, 141.4, 128.0, 118.0, 109.2, 100.7, 77.0, 66.5, 19.5.

### Synthesis of Compound 1k

Compound 1k was prepared as a yellow solid following General Procedure 1 using 1-bromo-2-butanone as the alkyl bromide. 1-(2-amino-5-nitrophenoxy)butan-2-one (1k): Yield: 0.9 g (93%). ¹H NMR (400 MHz, DMSO-d6) δ 7.76 (dd, J=8.6 Hz, 2.4 Hz, 1H), 7.62 (d, J=2.4 Hz, 1H), 6.70 (d, J=8.8 Hz, 1H), 6.36 (s, 2H), 5.24 (s, 2H), 2.58 (q, J=8.8 Hz, 2H), 1.12 (t, J=6.9 Hz, 3H).

### Synthesis of Compound 2k (N₁₁)

Compound 2k was prepared as a dark brown solid from compound 1k following General Procedure 2. 1-(2,5-diaminophenoxy)butan-2-one, (2k): Yield: 0.58 g (58%). ¹H NMR (400 MHz, DMSO-d6) δ 6.40 (d, J=8.0 Hz, 1H), 6.32 (d, J=2.0 Hz, 1H), 5.92 (dd, J=8.4 Hz, 2.0 Hz, 1H), 4.95 (s, 2H), 5.02 (s, 2H), 2.36 (q, J=8.6 Hz, 2H), 1.14 (t, J=7.4 Hz, 3H). ¹³C NMR (400 MHz, DMSO-d6) 207.0, 148.0, 141.0, 128.0, 118.5, 109.7, 100.4, 80.0, 32.0, 8.2.

### Synthesis of Compound 11

Compound 11 was prepared as a yellowish brown solid following General Procedure 1 using 2-bromopropene as the alkyl bromide. 4-nitro-2-(prop-1-en-2-yloxy)aniline (11): Yield: 1.5 g (88%). ¹H NMR (400 MHz, DMSO-d6) δ 7.72 (dd, J=8.8 Hz, 2.4 Hz, 1H), 7.58 (d, J=2.4 Hz, 1H), 6.76 (d, J=8.8 Hz, 1H), 6.54 (s, 2H), 4.15 (s, 1H), 4.48 (s, 1H), 1.92-1.96 (m, 3H).

### Synthesis of Compound 21 (N₁₂)

Compound 21 was prepared as a brown solid from compound 11 following General Procedure 2. 2-(prop-1-en-2-yloxy)benzene-1,4-diamine, (21): Yield: 0.57 g (57%). ¹H NMR (400 MHz, DMSO-d6) δ 6.28 (d, J=8.0 Hz, 1H), 6.10 (d, J=2.0 Hz, 1H), 5.50 (dd, J=8.4 Hz, 2.0 Hz, 1H), 4.76 (br, 4H), 4.50 (s, 1H), 4.10 (s, 1H), 1.90-1.93 (m, 3H). ¹³C NMR (400 MHz, DMSO-d6) 158.5, 143.0, 141.2, 129.5, 117.5, 111.0, 103.0, 90.5, 21.5.

### Synthesis of Compound 1m

Compound 1m was prepared as a yellow-orange solid following General Procedure 1 using 2-bromo-N-methylethanamine as the alkyl bromide. 2-(2-(methylamino)ethoxy)-4-nitroaniline (1m): Yield: 1.1 g (85%). ¹H NMR (400 MHz, DMSO-d6) δ 7.84 (dd, J=8.8 Hz, 2.4 Hz, 1H), 7.66 (d, J=2.4 Hz, 1H), 6.84 (d, J=8.8 Hz, 1H), 6.40 (s, 2H), 5.58 (s, 1H), 4.20 (t, J=6.8 Hz, 2H), 2.94 (t, J=7.2 Hz, 2H), 3.32 (t, J=7.2 Hz, 3H).

### Synthesis of Compound 2m (N₁₃)

Compound 2m was prepared as a dark brown solid from compound 1m following General Procedure 2. 2-(2-(methylamino)ethoxy)benzene-1,4-diamine, (2m): Yield: 0.61 g (61%).

¹H NMR (400 MHz, DMSO-d6) δ 6.65 (d, J=8.0 Hz, 1H), 6.29 (d, J=2.0 Hz, 1H), 5.65 (dd, J=8.4 Hz, 2.0 Hz, 1H), 4.72 (br, 4H), 5.48 (s, 1H), 4.12 (t, J=6.8 Hz, 2H), 2.87 (t, J=7.2 Hz, 2H), 3.20 (t, J=7.2 Hz, 3H). ¹³C NMR (400 MHz, DMSO-d6) 145.5, 140.3, 127.2, 117.4, 108.5, 100.0, 68.5, 51.5, 36.0.

### Example 8: Solubility Determination of Hair Dyes and Comparison with PPD Compounds

Solubility profile of the synthesized compounds were compared with previous libraries of chemicals as described in WO 2019/098948.

### Thermodynamic Solubility Assay

The aqueous solubilities (pH=7) of these compounds were measured using the modified shake flask method and LC-MS/MS. Each hair dye was added into a 2 mL glass vial containing Milli-Q water (1 mL) to form the precipitates at 25° C. Then the mixture was subjected to a solubility-equilibrium stage. The vials were shaken at 300 rpm at 25° C. for 24 h. The precipitate was separated by centrifugation at 23,000 g for 20 min. Subsequently, 0.5 mL of supernatant was transferred into a 1 mL Eppendorf tube, and it was centrifuged again as mentioned above. The supernatant was then used for LC-MS/MS analysis. An Agilent 1290 Infinity ultra-high pressure liquid chromatography (UHPLC) binary pump, auto sampler, vacuum degasser, and column oven (Agilent Technologies Inc., Santa Clara, CA, USA) and ACQUITY UPLC BEH C18, 1.7 μM, 2.1×100 mm column (Waters, Mildord, MA, USA), were used for chromatographic separations.

The mass spectrometric analysis was performed by use of a AB SCIEX QTRAP 5500 tandem mass spectrometry (MS/MS) system (AB SCIEX, Framingham, MA, USA) operating in triple quadrupole positive mode (ESI+) equipped with an AB Sciex Turbo Ion Spray interface. Acquisition and analysis of data were performed with Analyst software ver. 1.4.2 (Applied Biosystems) which performed all chromatographic peak integration. For each hair dye, a standard curve consisting of four concentrations was established.

Solvent A was composed of 0.1% [v/v] formic acid in Milli-Q water while solvent B was composed of 0.1% [v/v] formic acid in acetonitrile. A mobile phase gradient pumped at 0.6 mL/min was used to elute the hair dyes from the column. HPLC gradient profile program used for elution is listed in the Table 2. The column was equilibrated for 1 min resulting in a total run time of 5 min. The injection volume was 5.0 μL for all derivatives. To prevent compound accumulation on the needle, 50% methanol in ACN was used as needle wash for 30 s per sample.

TABLE 2
HPLC gradient profiles for N1-N13 using
ACQUITY UPLC BEH C18 column.
Gradient Profile

| Time (min) | A % |
|---|---|
| 0.1 | 95.0 |
| 1.0 | 60.0 |
| 2.0 | 60.0 |
| 2.5 | 40.0 |
| 3.0 | 2.0 |
| 4.0 | 2.0 |
| 4.1 | 95.0 |
| 5.0 | 95.0 |

### Results

The synthesized compounds N1-N13 displayed exemplary solubility profile as compared to the previous series chemicals PPD 1-PPD 16. Among the PPD series, none of the chemicals showed water solubility (Table 4). In contrast, N1-N13 showed higher solubility profile than PPD from 32-100 mg/ml (Table 3). Overall, the novel compounds N1-N13 showed 1.5 to 2.5 times higher solubility as compared to PPD thus confirming hydrophilic nature of the series. Compounds N1, N2, N5, N6, N8, N10 showed solubility of 8.5 to 10% (78-100 mg/mL) compared to 4% for PPD (40 mg/mL). Compounds N3, N4, N7 and N9 displayed a solubility of about 6.5% to 7.2% (65 to 72 mg/mL). In contrast, compound N12 and N13 showed lower solubility profile than PPD, which without being bound by theory is believed to be due to the hydrophobic nature of chemicals.

TABLE 3
Solubility data of synthesized compounds. # cLogP: calculated LogP using
Chemdraw Professional 15.0, #Molar Volume calculated using ACD/Chemsketch version 14.0.

| | | | | ^{#} Molar | |
|---|---|---|---|---|---|
| | | Molecular | | Volume | Solubility |
| Code | —R′ | Weight | ^{#}cLog P | (cm³) | (mg/ml) |
| N1 | —CH₂(CH)(OCH₃)₂ | 212.25 | −0.425 | 180.6 + 3.0 | 85 + 4.0 mg/ml |
| N2 | —CH₂OCH₃ | 168.20 | −0.485 | 140.8 + 3.0 | 90 + 2.6 mg/ml |
| N3 | —CH₂OCH₂CH₃ | 182.22 | −0.096 | 157.3 + 3.0 | 65 + 3.6 mg/ml |
| N4 | 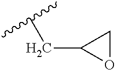 [SMILES: *CC1CO1] | 180.21 | −0.428 | 138.3 + 3.0 | 70 + 4.6 mg/ml |
| N5 | —CH₂CH₂OCH₂CH₂OCH₃ | 226.28 | −0.237 | 196.7 + 3.0 | 85 + 2.5 mg/ml |
| N6 | —CH₂CN | 163.18 | −0.959 | 127.8 + 3.0 | 100 + 3.0 mg/ml |
| N7 | —CH₂CH₂OCH₃ | 182.22 | −0.1022 | 157.3 + 3.0 | 62 + 2.0 mg/ml |
| N8 | —CH₂OCH₂OH | 184.20 | −0.7592 | 138.3 + 3.0 | 92 + 3.0 mg/ml |
| N9 | —CH₂CH₂OH | 168.20 | −0.865 | 132.0 + 3.0 | 72 + 3.4 mg/ml |
| N10 | —CH₂CH(OH)CH₃ | 182.22 | −0.556 | 148.8 + 3.0 | 78 + 2.5 mg/ml |
| N11 | —CH₂COCH₂CH₃ | 194.23 | −0.11 | 164.6 + 3.0 | 45 + 4.1 mg/ml |
| N12 | —C(═CH₂)CH₃ | 164.21 | 1.132 | 145.9 + 3.0 | 32 + 3.7 mg/ml |
| N13 | —CH₂CH₂NHCH₃ | 181.24 | −0.486 | 159.8 + 3.0 | 38 + 2.8 mg/ml |
| | PPD | 108.14 | −0.312 | 93.9 + 3.0 | 40 + 3.5 mg/ml |

All values were obtained from 3 separate determinations.

TABLE 4
Chemical structure, solubility data of library of old series.

| | | Water |
|---|---|---|
| PPD | | Solubility |
| derivatives | Structure | (mg/ml) |
| PPD 1 | 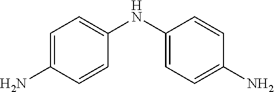 [SMILES: Nc1ccc(Nc2ccc(N)cc2)cc1] | 0.5 mg/ml |
| PPD 2 | 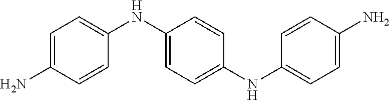 [SMILES: Nc1ccc(Nc2ccc(Nc3ccc(N)cc3)cc2)cc1] | 0.1 mg/ml |
| PPD 3 | 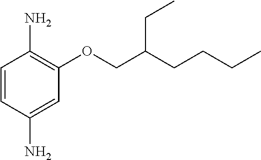 [SMILES: CCCCC(CC)COc1cc(N)ccc1N] | 0.08 mg/ml |
| PPD 4 | 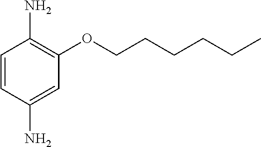 | 0.08 mg/ml |
| PPD 5 | 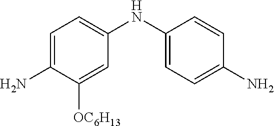 | Insoluble |
| PPD 6 | 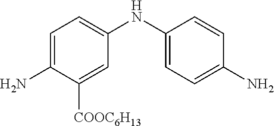 | 0.001 mg/ml |
| PPD7 | 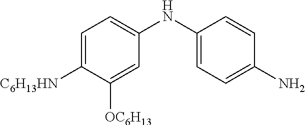 | 0.005 mg/ml |
| PPD8 | 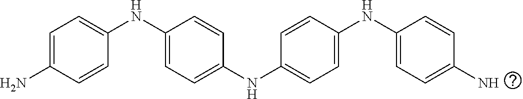 [SMILES: Nc1ccc(Nc2ccc(Nc3ccc(Nc4ccc(N)cc4)cc3)cc2)cc1] | Insoluble |
| PPD9 | 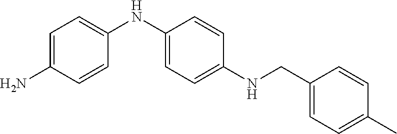 [SMILES: Cc1ccc(CNc2ccc(Nc3ccc(N)cc3)cc2)cc1] | Insoluble |
| PPD10 | 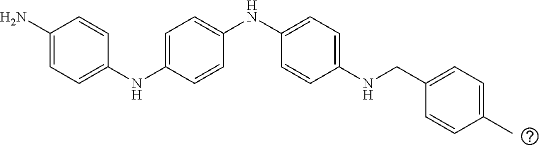 [SMILES: Cc1ccc(CNc2ccc(Nc3ccc(Nc4ccc(N)cc4)cc3)cc2)cc1] | Insoluble |
| PPD11 | 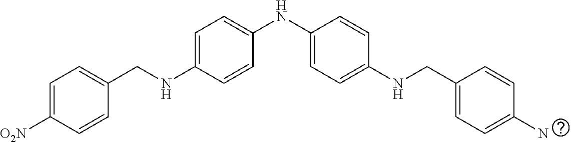 [SMILES: Nc1ccc(CNc2ccc(Nc3ccc(NCc4ccc([N+](=O)[O-])cc4)cc3)cc2)cc1] | Insoluble |
| PPD12 | 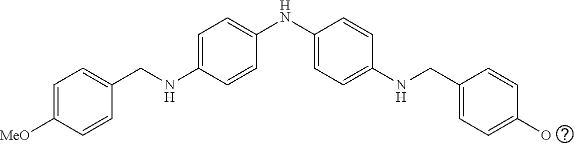 [SMILES: COc1ccc(CNc2ccc(Nc3ccc(NCc4ccc(O)cc4)cc3)cc2)cc1] | Insoluble |
| PPD13 | 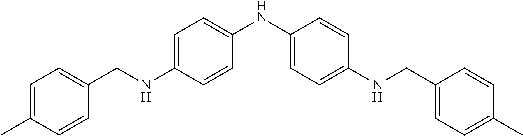 | Insoluble |
| PPD14 | 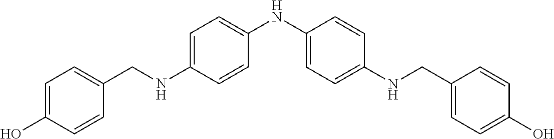 [SMILES: Oc1ccc(CNc2ccc(Nc3ccc(NCc4ccc(O)cc4)cc3)cc2)cc1] | Insoluble |
| PPD15 | 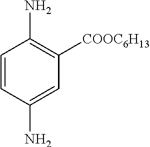 | 0.010 mg/ml |
| PPD16 | 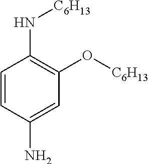 | Insoluble |
| PTD (2- methylbenzene- 1,4-diamine) | 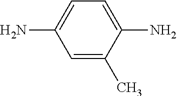 | 2.5 mg/ml |
| ME-PPD 2- (methoxymethyl) benzene- 1,4-diamine) | 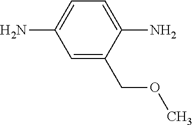 | 78 mg/ml |

### Example 9: MTT Assay on HaCaT Cells

### Method

HaCaT (human, adult, low calcium, high temperature, skin keratinocyte) cell suspension was adjusted to 5×10³ cells per well and seeded in 96-well plates, then incubated at 37° C. with 5% CO₂. After 24 h, 10 mM stock solutions of N1-N13 in autoclaved milli-Q water were prepared and serially diluted concentrations were added to the wells. Each concentration was performed in 6-replicates, and seeded wells with only media and no compound were used as controls while wells with only media were used as blanks. The plate was incubated for 72 h, after which 100 μL of MTT reagent was added and the plate was further incubated for 3 h before adding 100 μL of DMSO. Subsequently, the plate was shaken for 20 min, and then absorbance at 570 nm was determined using a Bio-Tek plate reader.

### Results

Error! Reference source not found. A & 1B show the percentage cell viability of HaCaT cells after being exposed to various concentrations of N1-N13. Table 5 is a comparison of the cytotoxicity profile of N1-N13 with published data PPD, ME-PPD and PTD (J Hazard Mat., 2021, 402, 123712). Simvastatin was selected as positive control due to its well-documented cytotoxic effect on HaCaT cells. The findings show that the introduction of an electron-donating —O—R′ group substituted on the ortho position of PPD can improve cytotoxicity of the compound. All derivatives N1-N13 displayed better cytotoxicity profile with higher IC₅₀ values than PPD (IC₅₀=23.45 μM).

TABLE 5
IC₅₀ values of N1-N13.

| Hair Dye | IC₅₀/μM |
|---|---|
| PPD^{#} | 23.45 |
| ME-PPD^{#} | 334.5 |
| PTD^{#} | 73.13 |
| N1 | 508.3 |
| N2 | 297.1 |
| N3 | 457.0 |
| N4 | 578.0 |
| N5 | 43.3 |
| N6 | >1000 |
| N7 | 33.5 |
| N8 | 218.9 |
| N9 | 271.6 |
| N10 | 282.8 |
| N11 | 195.6 |
| N12 | 127.4 |
| N13 | 120.4 |
| Simvastatin^{#} | 9.27 |
| (Positive Control) | |

^{#}indicates data from J Hazard Mat., 2021, 402, 123712.

### Example 10: Direct Peptide Reactivity Assay

### DPRA Incubation

The assay was conducted on N1-N13 in triplicates with the respective solvent used to prepare compound stock solutions as control. A stock concentration of 20 mM was prepared instead of the recommended 100 mM by OECD due to scarcity of compounds. The various constituents of the incubation mixture were freshly prepared to prevent stability issues that could cause variations in results. An incubation mixture with the recommended peptide to test compound ratios of 1:10 for cysteine heptapeptide and 1:50 for lysine heptapeptide (OECD. Test No. 442C: In Chemico Skin Sensitisation 2020) was prepared then incubated at 25° C. for 24 h in a shaking incubator (Toxicol Sci. 2012; 129(2):421-31).

At 24 h, the incubation mixture was quenched with 75 μL of 95% ACN/H₂O containing 200 μM Leucine Enkephalin (Internal Standard). For cysteine DPRA, 100 μL of quenched mixture was spiked into 90 μL of 2% ACN/H₂O and 10 μL of freshly prepared 0.016 mM 1,4-Dithiothreitol (DTT) solution. DTT maintains the integrity of thiol groups by preventing the dimerization of thiol groups and degradation of di-adducts to mono-adducts. This second mixture was then incubated at 40° C. for 30 min in a shaking incubator. For lysine DPRA, 10 μL of quenched mixture was spiked into 190 μL of 2% ACN/H₂O. All 200 μL of mixtures were transferred into a 96-well plate and sent for analysis with Liquid Chromatography-Tandem Mass Spectrometry (LC-MS/MS).

### Results

The DPRA results of N1-N13 all revealed a lower mean peptide depletion percentage (i.e. lower sensitizing potential) in comparison with PPD. N1-N4, N6 and N8-N10 showed weak sensitizing potential with lowest mean peptide depletion values (17.2±2.3% to 22.5±1.9%, respectively). N5, N7 and N11-N13 showed moderate sensitizing potential when compared to the commercially available PPD, ME-PPD and PTD.

It can be observed that R groups with longer chains and more diether substituents (e.g. N5) and highly electrophilic alkene handle (e.g. N12) showed higher reactivity to nucleophilic peptides and therefore are less favourable groups as the R′ substituents on the ortho position. N12, having a substituent alkenyl sidechain, showed the highest reactivity (43.7±4.0%). In contrast, N6 which was substituted with CN showed the lowest depletion and N4 with an epoxide group had slightly higher peptide reactivity than N6. Two structurally similar and same molecular weight compounds N3 and N7 displayed a different profile to each other. N8-N10, having a hydroxyl group on the side chain, showed consistently lower percent depletion from 19.5±3.6% to 22.5±3.8%. In contrast, N11 with a ketone side chain and N13 with an ethylamine side chain did not improve the peptide depletion and remained moderate sensitizers. N3 emerged as one of the best molecules with sensitization potential of almost 17% as compared to N7 that displayed moderate sensitization (30.4±2.9%) capability. One reason for their activity difference could be due to the presence of the O-ethyl chain. Similar observation is noticed with least effective compound N5, again substituted with O-ethyl and further elongated with another etheric O-ethyl chain. This suggests that these O-ethyl linkages may be detrimental, perhaps due to their higher hydrophobicity than O-methyl chain compounds (N1-N4 and N6). The results also suggest that the presence of a hydroxyl group on the side chain is favorable for the activity. Nevertheless, a skilled person will appreciate that all of compounds N1-N13 displayed reduced sensitization than existing compounds, and all of N1-N13 are advantageous over PPD, MEPPD and PTD.

### Example 11: Skin Permeation Study

Franz Diffusion Cell method

Skin Permeation study was conducted as previously reported (New Journal of Chemistry. 2019; 43(41):16188-99; Journal of Hazardous Materials. 2021; 402). In each Franz cell, a piece of pig ear skin prepared to a thickness of −1 mm was clamped securely. 5 mL of PBS solution was added to the receiver compartment which was purged of air bubbles to ensure maximal contact of the skin piece with the solution. Donor solution was prepared by dissolving N1-N13 in deionized water at 1% w/v, and 900 μL was added to the donor compartment. The study was conducted for 8 h, and aliquots of 200 μL were removed from the receiver solution and topped up with fresh PBS at each timepoint of 15-minutes intervals for the first hour and every hour after that.

For mass balance, at the end of 8 h, skin pieces and swabs of the top and bottom surface of the skin were collected and stored in ACN for N1-N13 extraction. Donor solution and receiver solution were also collected.

### Sample Processing and Analysis

The aliquot samples collected were subjected to 2-step extraction using 750 μL of dichloromethane (DCM). To improve extraction efficiency, 40 μL of 25% ammonium hydroxide was added to convert the compounds into Lewis bases. The solvent was dried using the TurboVap, and then reconstituted with 198 μL of 0.1% formic acid in 50% methanol in water and spiked with 2 μL of ANP (internal standard). The processed aliquot samples and mass balance samples were then sent for analysis using LC-MS/MS. The compounds' steady state fluxes and permeability coefficients were then calculated by fitting the solution to Fick's second law of diffusion at the steady state (linear part) of each experimental cumulative amount curve.

### Results

Skin permeation of N1-N13 was assessed using porcine ear skin inserted in Franz diffusion cells. The receptor compartment of the Franz cells was assayed at different time points (0-8 h) following application of the compound to the skin, to obtain the cumulative amount penetrated over time. Samples were collected from porcine skin permeation study and stored at −80° C. FIGS. 5i and 5ii summarise the extracted Ion chromatogram (XIC) of N1-N13 as derived from porcine skin permeation study. Table 6 summarizes the mean cumulative amount of N1-N13 at 8 h after application. A higher mean cumulative amount suggests greater penetration (i.e. higher permeability) of the compound into the skin. N1-N13 had significantly lower skin penetration compared to PPD.

TABLE 6
Cumulative amount of N1-N13 at 8^{th} hour and
mean percentage parent compound recovery.

| | Cumulative | Cumulative amount | |
|---|---|---|---|
| | amount at 8^{th} | comparison of PPD | Total |
| Hair Dye | hour (μg/cm²) | vs derivatives | recovery |
| PPD^{#} | 5.4 | — | 93 ± 0.52 |
| ME-PPD^{#} | 0.519 | 10.4 | 95 ± 0.79 |
| PTD^{#} | 0.287 | 18.8 | 99 ± 0.33 |
| N1 | 0.00730 | 740.0 | 96 ± 1.26 |
| N2 | 0.0119 | 453.7 | 98 ± 1.33 |
| N3 | 0.00875 | 617.0 | 97 ± 1.05 |
| N4 | 0.0104 | 520.0 | 99 ± 1.15 |
| N5 | 0.00654 | 825.0 | 95 ± 1.42 |
| N6 | 0.0145 | 372.4 | 99 ± 1.37 |
| N7 | 0.00800 | 675.0 | 97 ± 1.49 |
| N8 | 0.00843 | 640 | 95.1 ± 1.3 |
| N9 | 0.0121 | 445 | 94.6 ± 1.5 |
| N10 | 0.008 | 633 | 95.1 ± 1.4 |
| N11 | 0.00794 | 680 | 93.8 ± 1.3 |
| N12 | 0.0144 | 375 | 96.1 ± 1.0 |
| N13 | 0.0106 | 510 | 93.1 ± 1.2 |

^{#}indicates data from J Hazard Mat., 2021, 402, 123712.

The data are shown as percentages of the dose of compound applied to the skin. Skin permeation and amounts of N1-N13 recovered in the skin were almost three orders of magnitude less than those of PPD.

In general, the introduction of an —O—R group at the ortho position of PPD decreases permeability of N1-N13 to a significant extent. Almost all compounds were impermeable to the skin, possibly due to their larger molecular size. As expected, the permeation profile decreased with increasing molecular weight of the derivatives from N1-N13. The smallest derivative N6 (163.17 Da) and largest derivative N5 (226.3 Da), have molecular weights 1.5-fold and 2.0-fold greater than the molecular weight of PPD respectively. Other physico chemical parameters such as molar volume (Table 3) also play a pivotal role in skin permeability. As noticed with molecular weight, skin permeation of the compounds decreases with increasing molar volume. The smallest derivative N6 has a molar volume of 127.8 cm³, while the largest derivative N5 has higher molar volume of 196.7 cm³.

TABLE 7
Mass balances on N1-N13 (expressed as % of applied compound dose, means ± s.d.).

| | Permeated | | | | Recovered in | Recovered |
|---|---|---|---|---|---|---|
| | through | Recovered | | Recovered | skin outside | on parafilm |
| | skin into | in donor | Recovered | in skin | diffusion | and in |
| Compound | receptor | compartment | in skin | swabs | area | washes |
| PPD# | 0.038± | 83 ± | 4.3 ± | 0.82 ± | 0.0050± | 2.19± |
| | | 0.45 | 0.17 | 0.031 | | |
| ME- | 0.0044 ± | 89 ± | 3.6 ± | 0.69 ± | 0.0042 ± | 1.84 ± |
| PPD# | 0.0000 | 0.79 | 0.14 | 0.026 | 0.0003 | 0.0084 |
| PTD# | 0.0020± | 95 ± | 2.1 ± | 0.48 ± | 0.0077± | 0.68± |
| | | 0.31 | 0.021 | 0.016 | | |
| N1 | <0.001 | 96.7 ± | 0.05 ± | 0.04 ± | 0.0015 ± | 0.003 ± |
| | | 1.21 | 0.0076 | 0.008 | 0.0005 | 0.036 |
| N2 | <0.001 | 98.2 ± | 0.04 ± | 0.05 ± | 0.0024 ± | 0.004 ± |
| | | 1.33 | 0.003 | 0.0005 | 0.0006 | 0.003 |
| N3 | <0.001 | 97.2 ± | 0.01 ± | 0.04 ± | 0.004 ± | 0.005 ± |
| | | 1.05 | 0.0004 | 0.0007 | 0.0003 | 0.0021 |
| N4 | <0.001 | 98.9 ± | 0.02 ± | 0.03 ± | 0.005 ± | 0.008 ± |
| | | 1.14 | 0.004 | 0.0009 | 0.0004 | 0.0032 |
| N5 | <0.001 | 95.3 ± | 0.04 ± | 0.06 ± | 0.0045 ± | 0.009 ± |
| | | 1.42 | 0.0019 | 0.0001 | 0.0005 | 0.0014 |
| N6 | <0.001 | 99.1 ± | 0.01 ± | 0.04 ± | 0.006 ± | 0.002 ± |
| | | 1.30 | 0.005 | 0.0004 | 0.0007 | 0.005 |
| N7 | <0.001 | 97.5 ± | 0.03 ± | 0.09 ± | 0.007 ± | 0.001 ± |
| | | 1.48 | 0.006 | 0.0003 | 0.0002 | 0.0012 |
| N8 | <0.001 | 95.0 ± | 0.06 ± | 0.02 ± | 0.0018 ± | 0.004 ± |
| | | 1.4 | 0.004 | 0.006 | 0.0002 | 0.001 |
| N9 | <0.001 | 94.5 ± | 0.05 ± | 0.03 ± | 0.0022 ± | 0.003 ± |
| | | 2.1 | 0.004 | 0.0007 | 0.0003 | 0.002 |
| N10 | <0.001 | 95.0 ± | 0.04 ± | 0.05 ± | 0.003 ± | 0.006 ± |
| | | 1.25 | 0.0001 | 0.0008 | 0.0001 | 0.002 |
| N11 | <0.001 | 93.7 ± | 0.06 ± | 0.025 ± | 0.002 ± | 0.005 ± |
| | | 1.34 | 0.005 | 0.0003 | 0.0003 | 0.001 |
| N12 | <0.001 | 96.0 ± | 0.02 ± | 0.05 ± | 0.0045 ± | 0.007 ± |
| | | 1.18 | 0.003 | 0.0002 | 0.0004 | 0.001 |
| N13 | <0.001 | 93.0 ± | 0.03 ± | 0.02± | 0.003 ± | 0.003 ± |
| | | 1.5 | 0.004 | 0.0005 | 0.0005 | 0.001 |

#indicates data from J Hazard Mat., 2021, 402, 123712.

N1 (MW 212.25 Da) yielded a cumulative amount of 0.0073 μg/cm², about 740 times less than that of PPD. The cumulative amount of N2 (MW 168.20 Da) and N9 (MW 168.20 Da) penetrated was approximately 0.0120 μg/cm², or 445-450 times less than PPD. Among the O-alkyl etheric functional series, N3, N7 and N11 with similar molecular weight (MW 182.22 Da) displayed cumulative amount of 0.0088 μg/cm² and 0.0080 μg/cm², respectively. This is about 617- and 675-times lower permeation than PPD. Similarly, other compounds N4, N13 with close molecular weight to N3, N7 and N11 showed around 500 times less permeation. Further hydroxyl side chain compound N8 (0.00843 μg/cm²) displayed similar profile as N3-N4, and N7 with 640 times lower permeation. Another multi-etheric compound with longest chain substitution, N5 displayed the lowest cumulative amount at 8 h, 0.0065 μg/cm², which is almost 825 times lower than that of PPD. In contrast, N6 and N12 with shorter chain and greater molecular size yielded the lowest cumulative amount in the series, with 0.0145 μg/cm² around 370 times less permeation than standard PPD.

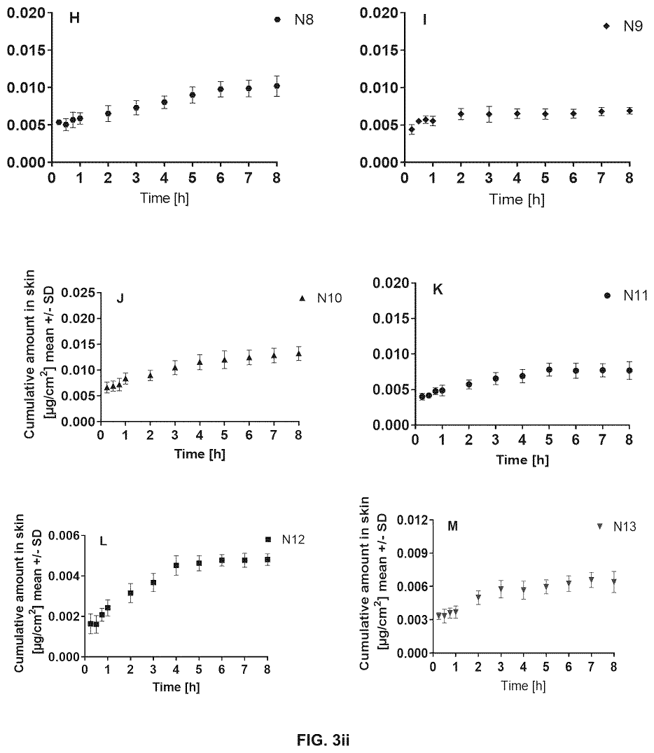

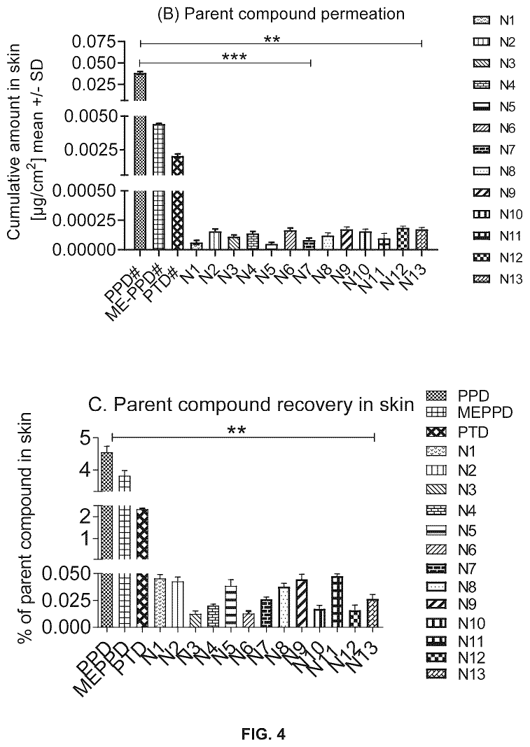

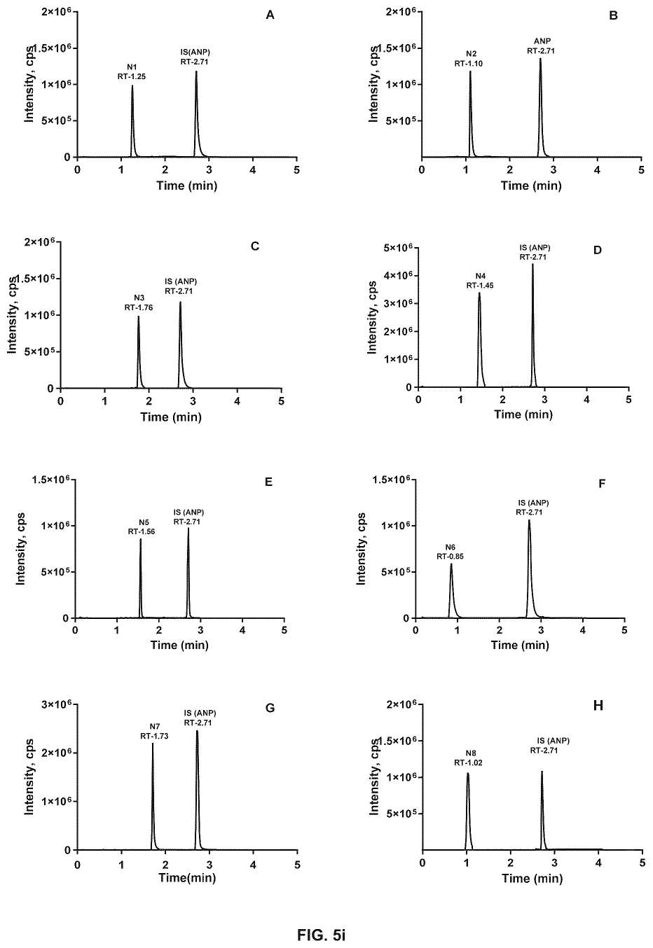

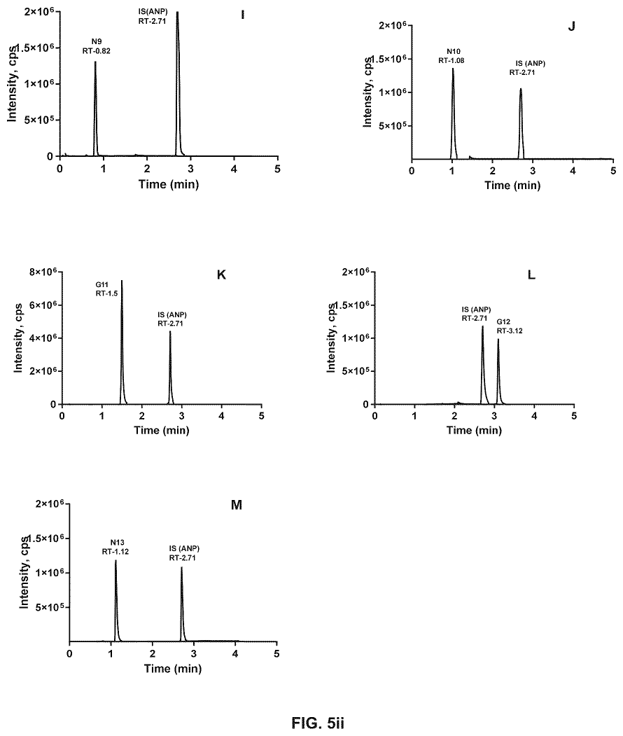

The corresponding cumulative amount versus time profiles (FIGS. 3i and 3ii) and mass balance results are provided in Table 7. Absorption of the N1-N13 derivatives into skin was significantly less than PPD in the skin. The amount of applied compound recovered at the end of the experiment ranged from 93% to 99% (FIG. 4A), indicating good mass balance. FIGS. 4B and 4C show, respectively, the percentages of applied dose permeated through the skin and recovered in the skin at the end of the permeation experiment.

### Example 12: LC-MS/MS Method for Detection of N1-N13

An Agilent 1290 Infinity ultra-high-pressure liquid chromatography (UHPLC) binary pump, auto sampler, vacuum degasser, and column oven (Agilent Technologies Inc., Santa Clara, CA, USA) and ACQUITY UPLC BEH C18, 1.7 μM, 2.1×100 mm column (Waters, Mildord, MA, USA), were used for chromatographic separations.

The mass spectrometric analysis was performed by use of an AB SCIEX QTRAP 5500 tandem mass spectrometry (MS/MS) system (AB SCIEX, Framingham, MA, USA) operating in triple quadrupole positive mode (ESI+) equipped with an AB Sciex Turbo Ion Spray interface. Acquisition and analysis of data were performed with Analyst software ver. 1.4.2 (Applied Biosystems) which performed all chromatographic peak integration. The temperatures of the analytical column and samples were maintained at 45° C. and 4° C. respectively. Solvent A was composed of 0.1% [v/v] formic acid in Milli-Q water while solvent B was composed of 0.1% [v/v] formic acid in acetonitrile. A mobile phase gradient pumped at 0.6 mL/min was used to elute the N1-N13 from the column. HPLC gradient program used for elution is listed in the Table 2. The column was equilibrated for 1 min resulting in a total run time of 5 min. The injection volume was 5.0 μL for all derivatives. To prevent compound accumulation on the needle, 50% methanol in ACN was used as needle wash for 30 s per sample.

### Preparation of Calibration Standards and Quality Control Samples

A stock solution of N1-N13 with a concentration of 10 mM was prepared by dissolving 2 mg of compounds in methanol. Working solutions were prepared by diluting the stock solutions of each analyte to a final concentration. Different stock standards were used to prepare quality control (QC) samples at the same concentrations. 200 μL of working calibrators (10 μM, 1 μM, 0.1 μM, 0.01 μM, 0.001 μM, 0.005 μM, 0.0005 μM, 0.0001 μM, 0.00001 μM) for N1-N13 were made in buffer (pH 7.4) medium. Low, medium and high quality control (LQC, MQC and HQC) samples for all were also prepared in buffer (Ph 7.4) medium at concentration of 7.5, 0.05 and 0.00025 μM using separate stock solutions. A working internal standard containing 50 μM of 2-amino-5-nitropyridine (ANP) was prepared by diluting the stock solutions of 2-amino-5-nitro pyridine (1.0 mg/mL) with methanol. Stock solutions and working solutions and standard solutions were stored at −20° C. until use.

### Tandem Mass Spectrometry

### Sample Processing and LC-MS/MS Analysis

TABLE 8
Chemical structures and respective multiple reaction monitoring
(MRM) transitions of 1. N1, 2. N2, 3. N3, 4. N4, 5. N5, 6.
N6, 7. N7, 8. N8, 9. N9, 10. N10, 11. N11, 12. N12, 13. N13,
14. 2-amino-5-nitropyridine (internal standard). The compound
dependent parameters were adjusted to provide the highest
sensitivity. The MS source conditions, and the compound-dependent
MS parameters are summarized in Table 9.

| | | RT | MRM |
|---|---|---|---|
| S. No | Analytes | (min) | Transition (m/z) |
| 1 | N1 | 1.25 | 213.0→134.8 |
| 2 | N2 | 1.10 | 169.0→127.8 |
| 3 | N3 | 1.76 | 183.1→124.0 |
| 4 | N4 | 1.45 | 181.4→135.0 |
| 5 | N5 | 1.56 | 227.2→95.0 |
| 6 | N6 | 0.85 | 165.4→80.0 |
| 7 | N7 | 1.73 | 183.3→95.2 |
| 8 | N8 | 1.02 | 184.2→154.1 |
| 9 | N9 | 0.82 | 168.2→168.2 |
| 10 | N10 | 1.08 | 182.2→124.1 |
| 11 | N11 | 1.5 | 194.2→164.2 |
| 12 | N12 | 3.12 | 164.2→138.1 |
| 13 | N13 | 1.12 | 181.2→167.2 |
| 14 | 2-amino-5-nitro | 2.71 | 140.1→94.0 |
| | pyridine | | |

TABLE 9
Compound-dependent MS parameters of N1-N13 and
2-amino-5-nitro pyridine (Internal standard).

| | DP | EP | CE | CXP | | | | | | |
|---|---|---|---|---|---|---|---|---|---|---|
| Derivatives | (V) | (V) | (V) | (V) | CUR | CAD | IS | TEM | GS1 | GS2 |
| N1 | 79.8 | 5.25 | 39.1 | 16.0 | 20 | High | 5500 | 500 | 50 | 55 |
| N2 | 34.1 | 9.73 | 7.9 | 9.1 | 15 | High | 5400 | 550 | 50 | 55 |
| N3 | 34.6 | 9.1 | 19.2 | 14.0 | 20 | High | 5400 | 550 | 55 | 50 |
| N4 | 133.9 | 8.3 | 27.2 | 19.1 | 20 | High | 5500 | 550 | 55 | 60 |
| N5 | 142.9 | 9.1 | 34.5 | 10.8 | 20 | Med | 5000 | 550 | 55 | 55 |
| N6 | 93.6 | 4.6 | 35.3 | 21.0 | 20 | Med | 5000 | 500 | 50 | 55 |
| N7 | 55.3 | 7.2 | 28.8 | 10.8 | 15 | High | 5400 | 500 | 50 | 55 |
| N8 | 105.4 | 6.3 | 29.6 | 17.4 | 18 | High | 5500 | 500 | 50 | 50 |
| N9 | 95.6 | 8.5 | 17.8 | 10.5 | 17 | High | 5400 | 500 | 50 | 50 |
| N10 | 125.3 | 7.5 | 24.6 | 12.7 | 16 | High | 5400 | 500 | 50 | 55 |
| N11 | 135.6 | 7.9 | 22.7 | 18.3 | 15 | High | 5500 | 500 | 55 | 55 |
| N12 | 146.8 | 9.5 | 31.8 | 12.5 | 20 | Med | 5000 | 500 | 55 | 55 |
| N13 | 86.5 | 5.6 | 30.4 | 20.5 | 20 | Med | 5000 | 500 | 50 | 50 |
| ANP (IS) | 65.0 | 8.0 | 23.0 | 15.0 | 15 | High | 5500 | 500 | 55 | 55 |

Abbreviations: DP: declustering potential, EP: entrance potential, CE: collision energy, CXP: collision exit potential, CUR: curtain gas, CAD: Collision gas (nitrogen), GS1: Ion source gas 1 (sheath gas), GS2: Ion source gas 2 (drying gas), IS: Ion spray voltage, Interface heater (Ihe) switched on; Quadrupole 1 and quadrupole 3 were maintained at unit resolution, Dwell time set was 100 ms for all compounds.

### Sample Preparation and Purification of Skin Permeation Study Samples for LC-MS/MS Analysis

Standards stock dilution was made in 100× concentration in methanol, i.e., Standard concentrations (Calibration standards and QC standards) of 1 mM, 100 μM, 10 μM, 7.5 μM, M, 1 μM, 0.1 μM, 0.5 μM, 0.05 μM, 0.01 μM, 0.001 μM, 0.025 μM of N1-N13 were prepared. 2 μL of each of the above concentrations were transferred into 2 mL Eppendorf tubes and the volumes made up to 200 μL using buffer (pH 7.4) media. For test samples, 200 μL of cell culture samples were directly used. 2 μL of an internal standard, 2-amino-5-nitro pyridine of concentration 100 μM was pipetted into the mixture. 40 μL of 28% NH₄OH was then added for efficient extraction of the compounds from buffer (pH 7.4) medium. To each tube, 1 mL of dichloromethane was added for extraction and each tube was vortexed for 3 mins. The aqueous layer was transferred into another set of 2 mL Eppendorf tubes and 1 mL of dichloromethane was further added for the second extraction of the compounds. After a second vortexing of each tube for 3 min and discarding of the aqueous layer, the two organic layers of dichloromethane were then combined and evaporated to dryness under a stream of nitrogen, using the TurboVap system at a pressure of 3-5 psi. Each standard and test solution sample was then reconstituted with 200 μL of the mobile phase, which is 1% formic acid in 50% methanol in water and was stored at 4° C. until LC-MS/MS.

### Example 13: MTT Assay and Cytokine IL-8 and IL-1α Analysis on THP-1 Leukemic Monocyte Cell Models

The next step is to prove if skin sensitization had occurred through further tests such as ELISA (enzyme-linked immunosorbent assay) analysis on cytokine IL-8, IL-1α. Before performing these tests, it is necessary to determine the CV75 (estimated concentration affording cell viability of 75%) values in N1-N13 of THP-1 dendritic cell models (Alternatives to Animal Testing and Experimentation. 2008; 13(2):70-82). This is because the CV75 value of THP-1 cells is used as the test dose in ELISA studies. IL-8 and IL-1α pro-inflammatory cytokines released in response to skin inflammation. The IL-8 and IL-1α receptor expression on skin keratinocytes is induced in response to inflammation. IL-8 and IL-1α acts as a chemokines to attract immune cells such as T-lymphocytes to prompt an immune response.

The inventors first determined the CV75 (estimated concentration affording 75% cell viability) values of N1-N13 in THP-1 cells.

### Method

### A. MTT Assay

THP-1 (human acute monocytic leukaemia cell line) cell suspension was adjusted to 1×10⁵ cells per well and seeded in 96-well plates. The plate was incubated at 37° C. with 5% CO₂. After 24 h, 10 mM stock solutions of N1-N13 were prepared in autoclaved milli-Q water. A series of concentrations diluted in media was prepared and added to the wells. Each concentration was performed in 6-replicates, and seeded wells with only media and no compound were used as controls while wells with only media were used as blanks. The plate was incubated for 24 h, after which 100 μL of MTT reagent was added and the plate was further incubated for 3 h before adding 100 μL of DMSO. Subsequently, the plate was shaken for 20 min, and then absorbance at 570 nm was determined using a Bio-Tek plate reader. Percentage cell viability was calculated and CV75 values were determined.

### B. IL-8 and IL-1α Release Assay (IL-8 and IL-1α Concentration Measured by ELISA)

CV75 values (concentration showing 75% of THP-1 cell survival) were determined as per the procedure described above in section A of this method. The IL-8 and IL-1α release assay was performed as described in Toxicol In Vitro. 2003, 17(3), 311-321. A Chemokine immunosorbent assay Bio-legend ELISA Max kit was used to quantify the levels of available IL-8 and IL-1α protein. The supernatant media of untreated THP-1 cells was treated with standards, test samples N1-N13 and DMSO in 96 well plate and then was recovered after 24 h of culture. IL-8 and IL-1α was measured by ELISA in 96-well microtiter plates according to the manufacturer protocol (Biolegend, ELISA MAX™, Singapore).

### Results

Table 10 displays the CV75 values of THP-1 cells when exposed to N1-N13. As seen, N6, N1, N3, and N8-N10 showed better cytotoxicity (i.e. higher CV75 values) on THP-1 cells, mimicking the same trend as HaCaT cells.

TABLE 10
CV75 values of THP-1 cells when exposed to PPD,
MEPPD, PTD, N1-N13 and DNCB (positive control).

| | CV75 Concentrations (μg/mL) | | | | | | | |
|---|---|---|---|---|---|---|---|---|
| Hair Dye/ | 1/1.2⁶ × | 1/1.2⁵ × | 1/1.2⁴ × | 1/1.2³ × | 1/1.2² × | 1/1.2 × | 1 × | 1.2 × |
| Standards | CV75 | CV75 | CV75 | CV75 | CV75 | CV75 | CV75 | CV75 |
| PPD | 21.9 | 26.3 | 31.5 | 37.8 | 45.4 | 54.5 | 65.4 | 78.5 |
| DNCB | 1.0 | 1.2 | 1.4 | 1.7 | 2.1 | 2.5 | 3.0 | 3.6 |
| SLS | 18.7 | 22.5 | 27.0 | 32.4 | 38.9 | 46.7 | 56.0 | 67.2 |
| MEPPD | 48.4 | 58.1 | 69.7 | 83.7 | 100.4 | 120.5 | 144.6 | 173.5 |
| PTD | 14.5 | 17.4 | 20.9 | 25.1 | 30.1 | 36.1 | 43.4 | 52 |
| N1 | 48.0 | 57.6 | 69.1 | 82.9 | 99.6 | 119.5 | 143.4 | 172.1 |
| N2 | 24.2 | 29.1 | 34.9 | 41.9 | 50.3 | 60.3 | 72.4 | 86.9 |
| N3 | 38.5 | 46.2 | 55.4 | 66.5 | 79.8 | 95.8 | 115 | 138 |
| N4 | 20.8 | 25 | 30.0 | 36.0 | 43.3 | 51.9 | 62.3 | 74.8 |
| N5 | 22.9 | 27.4 | 32.9 | 39.5 | 47.4 | 56.9 | 68.3 | 81.9 |
| N6 | 41.4 | 49.7 | 59.6 | 71.6 | 85.9 | 103.1 | 123.7 | 148.4 |
| N7 | 14.9 | 17.9 | 21.5 | 25.8 | 30.9 | 37.2 | 44.6 | 53.5 |
| N8 | 47.2 | 56.6 | 67.9 | 81.5 | 97.9 | 117.5 | 141 | 169.2 |
| N9 | 42.1 | 50.6 | 60.7 | 72.9 | 87.5 | 105 | 126 | 151.2 |
| N10 | 39.1 | 47.0 | 56.4 | 67.7 | 81.2 | 97.5 | 117 | 140.4 |
| N11 | 21.9 | 26.2 | 31.5 | 37.8 | 45.4 | 54.5 | 65.4 | 78.4 |
| N12 | 14.9 | 17.9 | 21.5 | 25.8 | 31.0 | 37..2 | 44.7 | 53.6 |
| N13 | 18.1 | 21.8 | 26.1 | 31.4 | 37.7 | 45.2 | 54.3 | 65.1 |

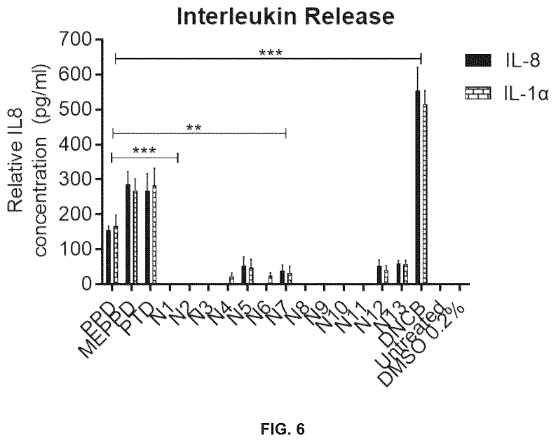

FIG. 6 displays the IL-8 and IL-1α elicitation profile of novel hair dyes (N1-N13) compared to hair dye standards like PPD, ME-PPD, PTD, positive (DNCB) and negative control (untreated, DMSO 0.2%). Relative IL-8 concentration (pg/ml) of various chemicals were quantified using standard curve. Skin sensitization potencies of novel derivatives were compared to hair dye standards such as PPD, ME-PPD, PTD, as well as positive (DNCB) and negative controls (untreated, DMSO 0.2%). Relative IL-8 and IL-1α concentrations (pg/ml) of various chemicals were quantified using a standard curve. Novel hair dyes N1-N4, N6 and N8-N11 untreated and DMSO 0.2% conditions did not release significant amounts of IL-8 and IL-1α cytokines. As expected, positive control DNCB exhibited 2-3 times higher release of IL-8 and IL-1α markers as compared hair dye standards. N4 displayed a slight release of IL-1α over IL-8 but 7-8 times less than PPD. N5, N7, N12, N13 showed slight elevation of both IL-8 and IL-1α levels. However, their level of release of IL-8 and IL-1α are advantageously 2-3 times lower than PPD. The hair dye standards PPD, MEPPD, PTD released higher concentrations of IL-8 and IL-1α. As expected, the positive control DNCB exhibited 2-3 times higher release of IL-8 and IL-1α markers as compared to hair dye standards. These results confirm that novel derivatives are advantageously non-sensitizers compared to standard hair dye.

### Example 14: hCLAT Assay CD86 and CD54 Expression Analysis by Flow Cytometry

### Method

The assay was conducted as per the literature (Toxicol In Vitro, 2006, 20, 767-73). THP-1 cells were cultured in 24-well plates (1×10⁶ cells/1 mL/well) with various concentrations of each chemical (N1-N13), +ve control (DNCB), −ve control for 24 h.

THP-1 cells were cultured in 24-well plates (1×10⁶ cells/mL/well) and treated with recommended concentrations of each chemical (N1-N13), positive control (DNCB) or negative control (SLS) for 24 h. THP-1 cells were cultured and treated with 8 doses of N1-N13 based on the CV75 namely, 1.2×CV75, 1×CV75, 1/1.2×CV75, 1/1.2²×CV75, 1/1.23×CV75, 1/1.24×CV75, 1/1.2⁵×CV75 and 1/1.2⁶×CV75. After 24 h, cells were transferred to 1.5 mL of microtubes and centrifuged at 450 g, 5 min, 4° C. Supernatant was discarded and cells were washed with 1 mL of FACS buffer (PBS+FBS+HEPES). The cells were centrifuged at 450 g, 5 min, 4° C. 50 μL of Fc block (0.01% of Globlins Cohn fraction II, III) was added and incubated for 10 min at 4° C. The cells were stained with 20 μL APC CD54 (Mouse IgG1, K, BD Biosciences, San Diego, CA, USA), 20 μL FITC CD86 (Mouse IgG1, K, BD Biosciences, San Diego, CA, USA) or their respective isotype controls and incubated for 15 min at room temperature in dark. After incubation, the antibodies were diluted with 200 μl FACS buffer. The cells were washed with 1 mL FACS buffer twice. Further, 200 μL Propidium iodide (PI, 0.625 μg/mL) prepared in FACS buffer was added before the flow cytometry analysis. Flow cytometric analysis was performed with BD LSR Fortessa™ (Becton Dickinson, San Jose, CA, USA) In total, 10,000 living cells were analysed, and data were processed using Flowjo software (v10.6, Ashland, OR, USA). The test concentration providing a cell viability of 75% (CV75) was derived from the dose response curve and was calculated by log-linear interpolation.

### Data Analysis and Prediction Model

Geometric mean fluorescence intensity (MFI) was measured for CD54⁺ and CD86⁺ cells separately and percentage relative fluorescence intensity (% RFI), reflective of CD86 and CD54 relative expression was calculated as described below. RFI was not calculated if cell viability fell below 50%.

$$\% RFI = \frac{\begin{matrix}MFI of chemical treated cells - MFI of \\ chemical treated isotype control cells\end{matrix}}{\begin{matrix}MFI of vehicle control cells - MFI of vehicle \\ isotype control cells\end{matrix}}$$

All chemicals were tested in three independent experiments. If in two of three independent experiments at any dose exceeded 150% RFI for CD86, or 200% RFI for CD54, the chemicals could be identified as a sensitizer. Otherwise it is identified as a non-sensitizer.

The data are expressed as the mean±standard error of mean (SEM). Student's t-test, one-way or two-way analysis of variance (ANOVA) followed by Tukey's post hoc test was used wherever applicable using GraphPad Prism version 8.0.1 (San Diego, CA, USA), p-value of ≤0.05 was considered statistically significant.

### Results

Since novel derivatives N1-N13 displayed noncytotoxic potential in HaCaT cells, nonreactive potential in DPRA and in vitro IL-8 and IL-1α assays, the inventors further conducted h-CLAT assay on THP-1 cells to investigate CD86 and CD54 marker expression to ascertain the skin sensitization potential of these derivatives. Positive control DNCB and negative control SLS were tested against standards PPD, ME-PPD and test chemicals N1-N13. Further, % RFI and Effective Concentrations (EC) values, such as EC150 for CD86 and EC200 for CD54, i.e. the lowest concentration at which the test chemicals induced a % RFI of 150 or 200, was calculated.

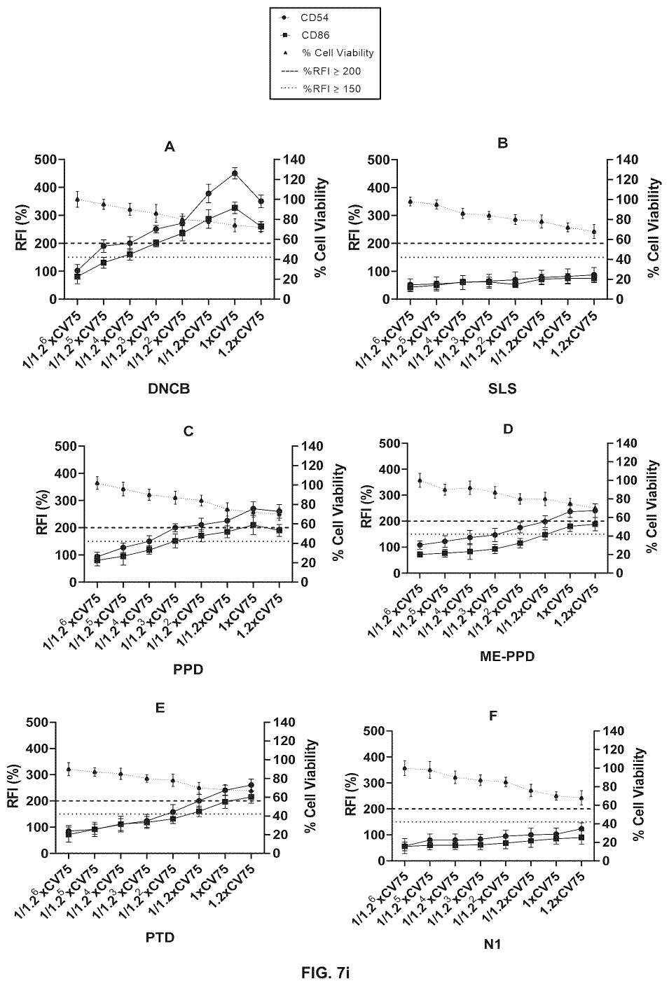

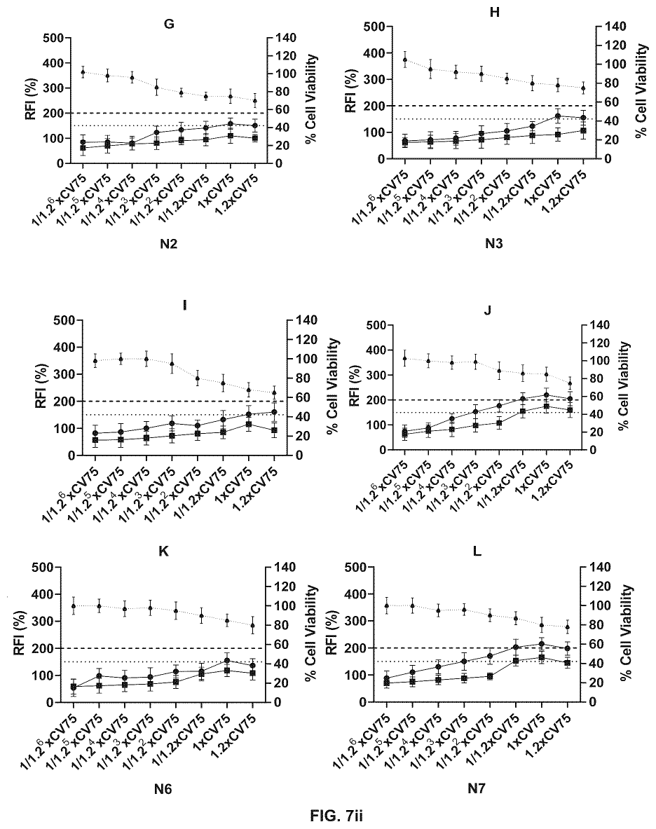

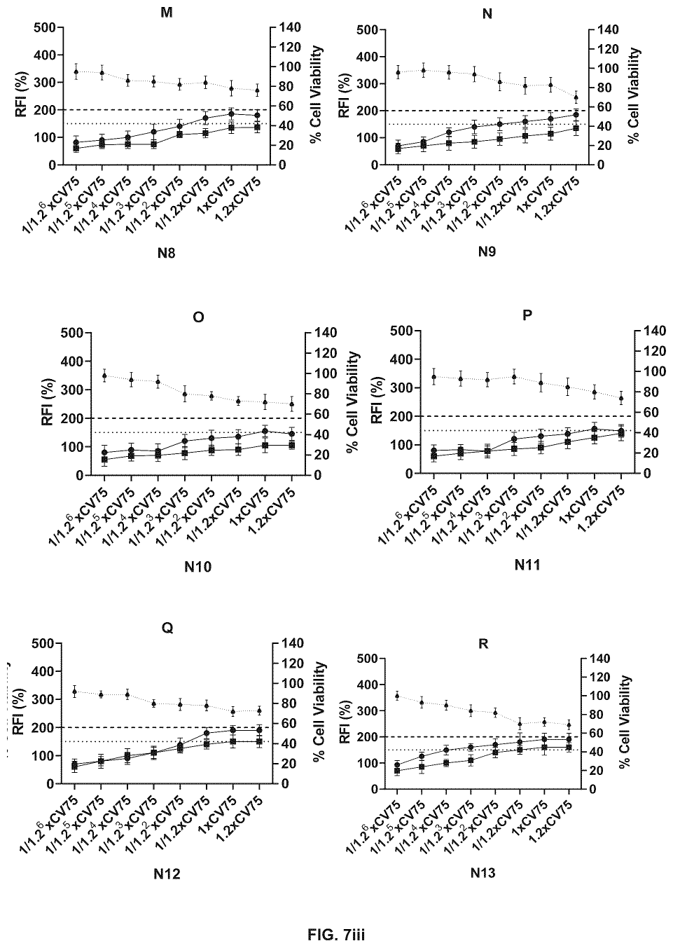

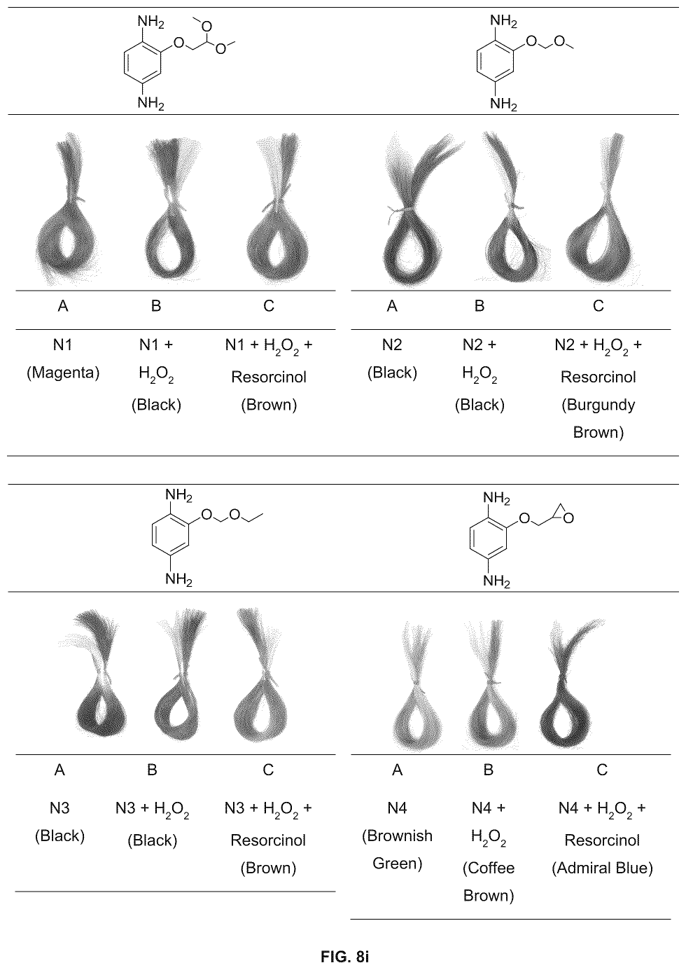

THP-1 cells were treated with increasing concentrations of DNCB, SLS, PPD, ME-PPD, PTD, N1-N7 and the CD54, CD86 expression was measured using flow cytometry. As shown in FIGS. 7i to 7iii, all the compounds (DNCB, SLS and N1-N13) displayed dose-dependent increase in the expression level of CD54 and CD86 markers. All the compounds displayed cell viability of more than 65% across all concentrations and were considered for further data analyses as per the OECD guideline. DNCB induced marker expression above OECD cut off from 1/1.2⁴×CV75 dose, peaked at CV75 and then declined. At CV75, DNCB expressed almost twice higher (RFI=450% for CD54, RFI=326% for CD86) than the OECD recommended range and displayed EC200, EC150 values of 1.5 μg/mL and 1.4 μg/mL respectively. As expected, the negative control SLS did not express markers, even at the highest concentration. Other standards PPD, MEPPD and PTD yielded CD54, CD86 expression above the recommended threshold. For example, PPD overexpressed markers from 1/1.2³×CV75 and sustained induction till CV75 dose and declined from 1.2×CV75. PPD followed a similar trend to DNCB, but 1.6 times lower expression in percentage at CV75 (RFI of 270% and RFI of 210% for CD54 and CD86 respectively) and displayed EC200, EC150 values of 35.0 μg/mL and 39.4 μg/mL respectively. The standard ME-PPD displayed slightly lower expression than PPD, but still decreased in the sensitizer range with EC200, EC150 values of 120.5 μg/mL and 117.9 μg/mL respectively. ME-PPD induced markers at CV75 dose and further elevated as dose level increased and A similar trend was noticed for PTD but markers induction noticed even at lower dose level 1/1.2×CV75 (EC200, EC150 values of 35.35 μg/mL and 34.3 μg/mL respectively) and expression trend fall in between PPD and ME-PPD.

Among the novel derivatives, N1, N4, N10 and N11 did not express the markers at any tested dose level and did not meet the OECD cut-off range for sensitizers. In addition, the induction was 3-4 times and 2-3 times lower than for DNCB and PPD, respectively. Expression levels were 150%-155%, 103% and 85%-125% for CD54 and CD86, respectively. These were only greater than those of the negative control SLS. N2, N3 and N6 followed a similar trend as N1 and N4, (RFI of approximately 155% and 160% for CD54 and 118% and 92% for CD86). This indicates 3 times lower induction than DNCB. Even within N series, N5, N7, N8, N9 and N12 failed to induce marker expression above the recommended % RFI cut-off (respectively 170% to 190% for CD54 and 115% to 145% for CD86). From this N5, N7 and N8-N9 and N12-N13 are deemed to be non-sensitizers. In summary, of all thirteen test compounds, none of them exhibited the skin sensitization potential in THP-1 cells at the tested concentrations.

### Example 15: Hair Nuance Test

### Bleached Hair Preparation

35 g of bleaching powder was mixed with 50 mL of developer then applied onto undyed black hair (wrapped in aluminium foil to enhance the bleaching process) and left on for 45 min. The hair was then rinsed with deionized water and shampoo and left to dry. This process was repeated twice.

### Hair Dye Formulations

Hair dye formulations were prepared from N1-N13 according to methods reported (New Journal of Chemistry. 2019; 43(41):16188-99), and with the compositions stated in Table 11 Bleached hair samples were immersed into the respective formulations for 20 min, then air dried for 20 min. Then they were shampooed twice and treated with conditioner for 5 min.

TABLE 11
Composition of Hair Dye Formulation.

| | Dye Composition | | | | | |
|---|---|---|---|---|---|---|
| | | | | | Type C | |
| Content of | Type A | | Type B | | (Coupler and | |
| Formulation | (Non-oxidative) | | (Oxidative) | | Oxidative) | |
| N1-N13 | 0.1 | g | 0.1 | g | 0.1 | g |
| Resorcinol | | | | | 0.1 | g |
| Deionized | 5 | mL | 4.70 | mL | 4.70 | mL |
| water | | | | | | |
| 30% H₂O₂ | | | 0.15 | mL | 0.15 | mL |
| pH adjusted with | | | pH 10-10.5 | | pH 10-10.5 | |
| 25% NH₄OH | | | | | | |
| Total Volume | 5 | mL | 5 | mL | 5 | mL |

### Hair Colour Measurement

SkinColorCatch (Delfin Technologies Ltd, Kuopio, Finland) was used to measure CIELAB tristimulus L*, a* and b* values of the dyed hair samples and baseline L₀, a₀, b₀ values from untreated hair. The difference in colour tone (ΔH) and the total colour difference (ΔE) were then derived from the values obtained with the equations below:

ΔH=\sqrt{(a*−a₀)²+(b*−b₀)²}

ΔE=\sqrt{(L*−L₀)²+(a*−a₀)²+(b*−b₀)²}

### Results and Discussion

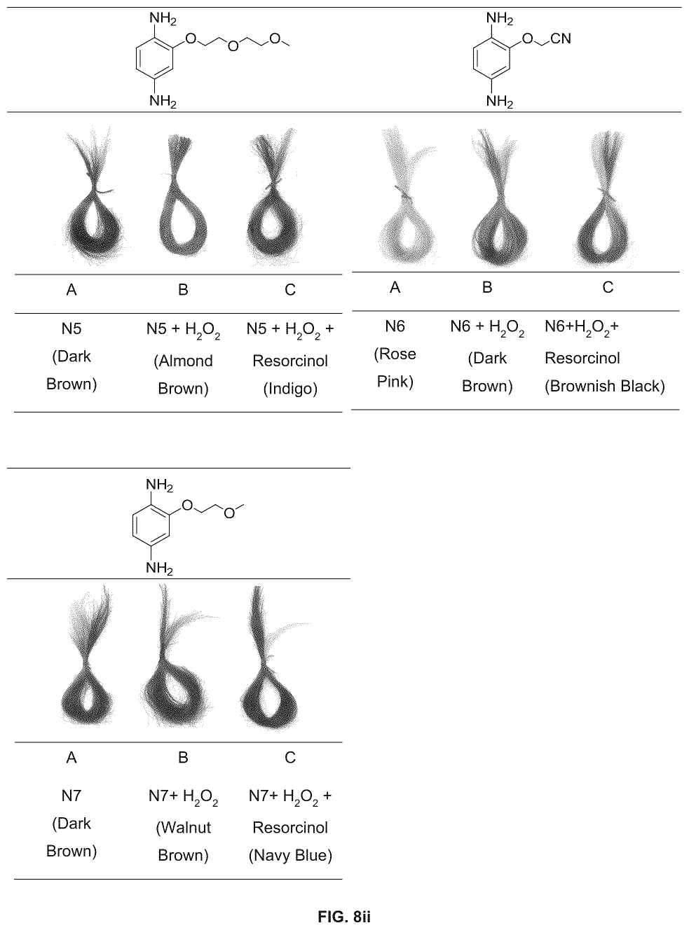

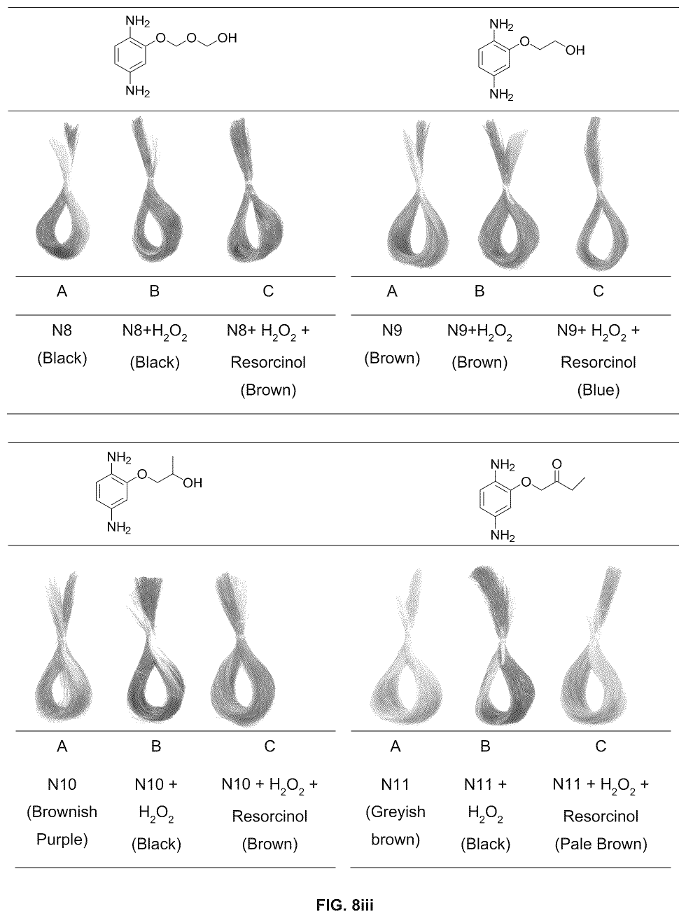

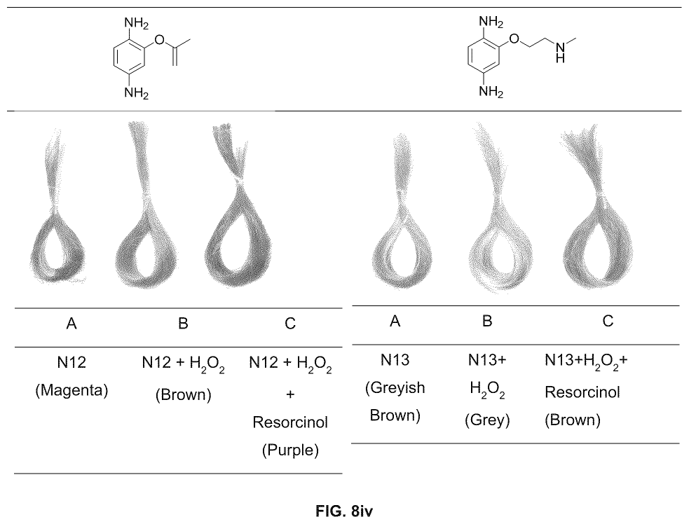

FIGS. 8i to 8iv show the pictures of eventual colours after N1-N13 derivatives were applied onto bleached hair samples. While the colours are not visible in black and white, the colour of each hair sample is provided in the FIG. 8i to 8iv. Table 12 describes the eventual colours of the bleached hair samples, and Table 13 shows their CIELAB Tristimulus values after performing Hair Nuance Test on N1-N13. The measurements from hair dye standards were obtained from published data (New Journal of Chemistry. 2019; 43(41):16188-99). Three formulations were prepared in this test: Type A, which comprises of no oxidising agent, was used to investigate if a compound was able to colour the hair without the use of oxidising agents. Type B, which contains oxidising agent hydrogen peroxide, was used as the hair dye formulation available commercially generally contains oxidising agents; Type C is an oxidising formulation with a coupler resorcinol which was used to investigate the compounds' performance with a coupler.

N1-N13 were designed to be more water soluble through the introduction of various hydrophilic substituents at the ortho position of PPD. The goal of improving water solubility of the compounds is to increase their hair dyeing efficacy by maximizing the amount of hair dye in the formulation that is in contact with the hair during the hair dyeing process.

FIGS. 8i to 8iv shows the pictures of eventual colours after N1-N13 derivatives were applied onto bleached hair samples, Table 12 describes the eventual colours of the bleached hair samples, and Table 13 shows their CIELAB Tristimulus values after performing the Hair Nuance Test on N1-N13. L*, a*, b* refer to values of treated hair and L₀, a₀, b₀ are baseline values measured from natural, untreated hair. The measurements of N1-N13 are compared to hair dye standards from published data (J Hazard Mat., 2021, 402, 123712). Three formulations were prepared: Type A, no oxidizing agent; Type B, with an oxidizing agent; Type C, with an oxidizing agent+resorcinol.

Type A formulation without an oxidizing agent conferred dark colours for N1-N3, N5 and N7 and light colours for N4, N6 and N13. Further, during the development of Type B and C formulations, no precipitation of the compounds was observed upon adjusting the pH between 10-10.5 with ammonia solution, proving complete miscibility of these compounds and aqueous solubility at different pH condition. N1-N13 dyes were able to impart colour onto the hair shaft even without the presence of an oxidising agent as seen in FIGS. 8i to 8iv. N1-N13 Type B formulations resulted in various shades of black to brown hair. Among the series, N1-N3, N5 and N7-N11 imparted dark shades to the hair. N1, N8, N10, N11 which are naturally dark brown colour chemicals, imparted black colour upon oxidation (ΔE=2 to 3) with hydrogen peroxide. Similarly, N2 and N3 synthesised as natural black colour with high water solubility, imparted black colour with ΔE=3.7 and 11.6, respectively, and even without oxidation (ΔE=2.0 and 9.9). N4, N6, N11 and N13 Type A formulation resulted in distinct lighter colour such as brownish green and rose pink to greyish brown respectively, whereas N1 reflected a brown as its physical form. Similarly, N5, N7 imparted dark brown colour shade to the hair indicating the similarities in structural features. On the other hand, Type A and Type B formulation of N9 and N12 displayed a brown colour.

Most of the chemicals displayed brown colour by coupling with resorcinol (Type C formulation). Interestingly, Type C formulations of N4, N5, N7 and N9 resulted in vibrant blue colours and type C of N12 showed distinct purple colour. N1-N13 resulted in hair nuance that were deeper shades than the comparators PPD, ME-PPD and PTD.

Further, nuance stability study was conducted. The hair samples dyed with N1-N13 were subjected to weekly washing up to 3 months and measuring of their CIELAB Tristimulus values during this stability study, and all the hair samples appeared to retain their original hair colour. This confirms that the new hair dyes provide permanent hair colour which, once inside the cortex, does not leave the hair shaft upon repeated washings.

TABLE 12
Hair dye composition and eventual colours after N1-N13
derivatives were applied onto bleached hair samples.

| Compounds | Type A | Type B | Type C |
|---|---|---|---|
| N1 | Magenta | Black | Dark brown |
| N2 | Walnut brown | Dark brown | Black |
| N3 | Black | Black | Brownish black |
| N4 | Brownish green | Coffee brown | Admiral blue |
| N5 | Dark cinnamon brown | Almond brown | Indigo |
| N6 | Rose pink | Dark brown | Brownish black |
| N7 | Dark hazelnut | Walnut brown | Navy blue |
| N8 | Black | Black | Brown |
| N9 | Brown | Brown | Blue |
| N10 | Brownish Purple | Black | Brown |
| N11 | Greyish brown | Black | Pale Brown |
| N12 | Magenta | Brown | Purple |
| N13 | Greyish Brown | Grey | Brown |

TABLE 13
Hair colour measurements of N1-N13 following dyeing with
Type A (Non-oxidative), Type B (Oxidative) and Type
C (Oxidative with coupler resorcinol) formulations.

| | Type | L* | L₀ | a* | a₀ | b* | b₀ | ΔE | ΔH |
|---|---|---|---|---|---|---|---|---|---|
| Natural Hair | | | 9 | | 0 | | 1 | | |
| N1 | A | 23 | | 12 | | 8 | | 19.7 | 13.9 |
| | B | 9 | | −0 | | −1 | | 2.0 | 2.0 |
| | C | 21 | | 18 | | 8 | | 22.7 | 19.3 |
| N2 | A | 11 | | 1 | | −2 | | 3.7 | 3.16 |
| | B | 9 | | 0 | | −1 | | 2.0 | 2.0 |
| | C | 21 | | 17 | | 6 | | 21.4 | 17.7 |
| N3 | A | 19 | | 5 | | 4 | | 11.6 | 5.8 |
| | B | 9 | | 0 | | −1 | | 2.0 | 2.0 |
| | C | 17 | | −1 | | 2 | | 8.1 | 1.4 |
| N4 | A | 49 | | 5 | | 29 | | 49.1 | 28.4 |
| | B | 40 | | 6 | | 16 | | 35.0 | 16.2 |
| | C | 22 | | 1 | | −13 | | 19.1 | 14.0 |
| N5 | A | 18 | | 4 | | −1 | | 10.0 | 4.5 |
| | B | 26 | | 3.7 | | 10.3 | | 19.7 | 10.0 |
| | C | 16 | | 0 | | −6.7 | | 10.4 | 7.7 |
| N6 | A | 55.0 | | 10.0 | | 19.0 | | 50.3 | 20.5 |
| | B | 15 | | 4 | | 5 | | 8.2 | 5.6 |
| | C | 13 | | 5 | | 4 | | 7.1 | 5.8 |
| N7 | A | 22 | | 2.7 | | 4.7 | | 13.8 | 4.6 |
| | B | 19 | | 4.7 | | 3 | | 11.2 | 5.1 |
| | C | 12.3 | | −0.7 | | −4 | | 6.0 | 5.0 |
| N8 | A | 12 | | −1 | | 6 | | 5.91 | 2.43 |
| | B | 9 | | 0 | | −1 | | 2 | 1.41 |
| | C | 10 | | 0 | | 3 | | 2.23 | 1.49 |
| NO | A | 23 | | 8 | | 10 | | 18.46 | 4.29 |
| | B | 32 | | 0 | | 4 | | 23.19 | 4.81 |
| | C | 29 | | 19 | | 19 | | 32.93 | 5.73 |
| N10 | A | 25 | | 12 | | 6 | | 20.61 | 4.53 |
| | B | 11 | | 1 | | −2 | | 3.74 | 1.93 |
| | C | 30 | | 18 | | 18 | | 32.46 | 5.69 |
| N11 | A | 36 | | 5 | | 8 | | 28.33 | 5.32 |
| | B | 9 | | 0 | | −1 | | 2 | 1.41 |
| | C | 32 | | 18 | | 25 | | 37.8 | 6.14 |
| N12 | A | 17 | | 9 | | 4 | | 12.4 | 3.52 |
| | B | 28 | | 18 | | 12 | | 28.39 | 5.32 |
| | C | 11 | | 0 | | −3 | | 4.47 | 2.11 |
| N13 | A | 24 | | 8 | | 8 | | 18.38 | 4.28 |
| | B | 36 | | 11 | | 18 | | 33.74 | 5.8 |
| | C | 25 | | 14 | | 11 | | 23.49 | 4.84 |

### CONCLUSION

In this study, improved novel hair dyes, N1-N13, were assessed for their water solubility, hair dyeing efficacy, cytotoxicity, skin penetration and sensitizing potential. N1-N13 were designed to be more water soluble by the addition of strategic hydrophilic functional groups in the ortho position of PPD. Careful structural modification improved their solubility by 6.5 to 10%.

The efficacy of dyes was first assessed in hair nuance test. Type B formulations of N1-N3, imparted black colour, suggesting the natural black hair colour (ΔE=2-9.9). Similarly, type A formulation of N1-N3, N5, N7, N8, N10-N11 imparted a dark colour, indicating the deeper dark shading nature of the compounds. Results of the in vitro skin permeation study show a lack of skin penetration, on account of their molecular sizes. Cytotoxicity assay results in HaCaT keratinocytes suggest that O-methyl moieties (N1-N4, N6 and N8-N12) are more favorable than O-ethyl moieties (N5 and N7). In addition, DPRA results further confirms that O-ethyl chain compounds (N5 and N7) were less conducive compared to O-methyl chain compounds (N1-N4, N6 and N8-N12).

Finally, skin sensitization assays were further assessed by evaluation of specific marker cytokines (IL-8 and IL-1α) and surface proteins (CD54 and CD86) in monocytic THP-1 cells. O-methyl chain compounds showed no induction of IL-8 and IL-1α expression in THP-1 cells while N5, N7 and N12, N13 showed marginal release. h-CLAT assay results complemented the DPRA, IL-8 and IL-1α results. None of the test compounds induced marker expression exceeding the OECD cut-off (>200% RFI for CD54, >150% RFI for CD86) and hence were deemed to be non-sensitizers.

In this study the inventors have confirmed the toxic effects of known allergens, PPD and PTD in vitro. ME-PPD, a supposedly non-toxic alternative to PPD, exhibited comparable or only marginally attenuated toxic and sensitizing properties compared to PPD. This confirms the observation of ME-PPD-induced ACD in clinical settings. In contrast, the inventors found that N1-N13 were excellent non-toxic substitutes for PPD and ME-PPD. They showed high aqueous solubility, low permeation, lower cytotoxicity in HaCaT cells, and low sensitizing potential. Further they effectively colour the hair even in the absence of an oxidizing agent. N2 and N3, N8-N10 were the most efficient at imparting colour. Further, N1-N4 and N6, N8-N10 were identified to be minimally reactive as compared to the contact allergen PPD. Overall, the compounds of the invention are less prone to elicit an allergic or other immune response in a user, with preserved dyeing properties on the hair shaft under non-oxidative or oxidative conditions.

In view of the improved performance, the inventors have surprisingly demonstrated that N1-N13 are safe, efficacious, and consumer-friendly 2-in-1 bottle products.

1. A compound of formula I: 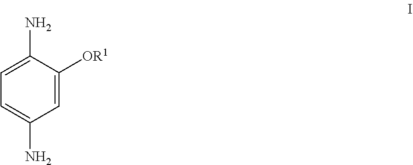
wherein:
R¹ represents C₁₋₁₀ alkyl substituted by one or more substituents selected from:
halo, OR², CN, ═O, ═CH₂, NHR², and C₁₋₃ alkyl, which latter group is unsubstituted or substituted by halo, CN, OR³; and
epoxy, provided that an epoxy substituent is only formed if R¹ is C₂₋₁₀ alkyl and two adjacent carbon atoms of R¹ together with an oxygen atom form the epoxy substituent,
R² represents H or C₁₋₅ alkyl, which is unsubstituted or substituted by one or more substituents selected from halo, OR⁴, and CN; and
R³ and R⁴ each independently represent H or C₁₋₃ alkyl, or a physiologically acceptable salt or solvate thereof.

2. The compound according to claim 1, wherein:
R¹ represents C₁₋₁₀ alkyl substituted by one or more substituents selected from:
halo, OR², CN, and C₁₋₃ alkyl, which latter group is unsubstituted or substituted by halo, CN, OR³; and
epoxy, provided that an epoxy substituent is only formed if R¹ is C₂₋₁₀ alkyl and two adjacent carbon atoms of R¹ together with an oxygen atom form the epoxy substituent,
R² represents H or C₁₋₅ alkyl, which is unsubstituted or substituted by one or more substituents selected from halo, OR⁴, and CN; and
R³ and R⁴ each independently represent H or C₁₋₃ alkyl.

3. The compound according to claim 1, wherein:
R¹ represents C₁₋₁₀ alkyl substituted by one or more substituents selected from:
halo, OR², CN, and C₁₋₃ alkyl, which latter group is unsubstituted or substituted by halo, CN, OR³; and
epoxy, provided that an epoxy substituent is only formed if R¹ is C₂₋₁₀ alkyl and two adjacent carbon atoms of R¹ together with an oxygen atom form the epoxy substituent,
R² represents C₁₋₅ alkyl, which is unsubstituted or substituted by one or more substituents selected from halo, OR⁴, and CN; and
R³ and R⁴ each independently represent C₁₋₃ alkyl.

4. The compound according to claim 1, wherein in the compound of formula I, R¹ represents C₁₋₆ alkyl, which is substituted with one or more substituents selected from OR², CN and epoxy, provided that an epoxy substituent is only formed if R¹ is C₂₋₆ alkyl and two adjacent carbon atoms of R¹ together with an oxygen atom form the epoxy substituent.

5. The compound according to claim 4, wherein in the compound of formula I, R¹ represents C₁₋₃ alkyl, which is substituted with one or more substituents selected from OR², CN and epoxy, provided that an epoxy substituent is only formed if R¹ is C₂₋₃ alkyl and two adjacent carbon atoms of R¹ together with an oxygen atom form the epoxy substituent.

6. The compound according to claim 5, wherein in the compound of formula I, R¹ represents C₁₋₃ alkyl, which is substituted with one or more substituents selected from CN and epoxy, provided that an epoxy substituent is only formed if R¹ is C₂₋₃ alkyl and two adjacent carbon atoms of R¹ together with an oxygen atom form the epoxy substituent.

7. The compound according to claim 1, wherein when present in the compound of formula I, R² represents C₁₋₃ alkyl, which is unsubstituted or substituted by one or more OR⁴ substituents.

8. The compound according to claim 7, wherein when present in the compound of formula I, R² represents C₁₋₂ alkyl, which is unsubstituted or substituted by one or more OR⁴ substituents.

9. The compound according to claim 1, wherein when present in the compound of formula I, R³ and R⁴ each independently represent methyl.

10. The compound according to claim 1, wherein:
R¹ represents C₁₋₄ alkyl substituted by one substituents selected from:
OR², ═O, ═CH₂, and NHR²;
R² represents H or C₁₋₂ alkyl which is unsubstituted or substituted by an OR⁴ group; and
R⁴ represents H or methyl.

11. The compound according to claim 1, wherein one or more of the following apply:
(a) R¹ is not substituted by C₁₋₃ alkyl, which C₁₋₃ alkyl is unsubstituted or substituted by halo, CN, OR³;
(b) R¹ represents C₁₋₆ alkyl, which is substituted with one or more substituents selected from halo, OR², CN, ═O, ═CH₂, NHR², and epoxy, provided that an epoxy substituent is only formed if R¹ is C₂₋₆ alkyl and two adjacent carbon atoms of R¹ together with an oxygen atom form the epoxy substituent;
(c) R¹ represents C₁₋₃ alkyl, which is substituted with one or more substituents selected from halo, OR², CN, ═O, ═CH₂, NHR², and epoxy, provided that an epoxy substituent is only formed if R¹ is C₂₋₃ alkyl and two adjacent carbon atoms of R¹ together with an oxygen atom form the epoxy substituent;
(d) when present, R² represents H or C₁₋₃ (e.g. C₁₋₂) alkyl, which is unsubstituted or substituted by one or more OR⁴ substituents;
(e) when present, R² represents H;
(f) when present, R⁴ represents H or C₁₋₂ alkyl;
(g) when present, R⁴ represents H;
(h) when present, R³ is not H.

12. The compound according to claim 1, wherein the compound of formula I is selected from the list:
(i) 2-(2,2-dimethoxyethoxy)benzene-1,4-diamine;
(ii) 2-(methoxymethoxy)benzene-1,4-diamine;
(iii) 2-(ethoxymethoxy)benzene-1,4-diamine;
(iv) 2-(oxiran-2-ylmethoxy)benzene-1,4-diamine;
(v) 2-[2-(2-methoxyethoxy)ethoxy]benzene-1,4-diamine;
(vi) 2-(2,5-diaminophenoxy)acetonitrile;
(vii) 2-(2-methoxyethoxy)benzene-1,4-diamine;
(viii) 2,5-diaminophenoxy)methoxy)methanol;
(ix) 2-(2,5-diaminophenoxy)ethan-1-ol;
(x) 1-(2,5-diaminophenoxy)propan-2-ol;
(xi) 1-(2,5-diaminophenoxy)butan-2-one;
(xii) 2-(prop-1-en-2-yloxy)benzene-1,4-diamine; and
(xiii) 2-(2-(methylamino)ethoxy)benzene-1,4-diamine, or
a physiologically acceptable salt or solvate thereof.

13. The compound according to claim 1, wherein the compound of formula I is selected from the list:
(i) 2-(2,2-dimethoxyethoxy)benzene-1,4-diamine;
(ii) 2-(methoxymethoxy)benzene-1,4-diamine;
(iii) 2-(ethoxymethoxy)benzene-1,4-diamine;
(iv) 2-(oxiran-2-ylmethoxy)benzene-1,4-diamine;
(v) 2-[2-(2-methoxyethoxy)ethoxy]benzene-1,4-diamine;
(vi) 2-(2,5-diaminophenoxy)acetonitrile; and
(vii) 2-(2-methoxyethoxy)benzene-1,4-diamine, or
a physiologically acceptable salt or solvate thereof.

14. (canceled)

15. The compound according to claim 13, wherein the compound of formula I is selected from the list:
(a) 2-(oxiran-2-ylmethoxy)benzene-1,4-diamine; and
(b) 2-(2,5-diaminophenoxy)acetonitrile;
a physiologically acceptable salt or solvate thereof.

16. The compound according to claim 12, wherein the compound of formula I is selected from the list:
(i) 2,5-diaminophenoxy)methoxy)methanol;
(ii) 2-(2,5-diaminophenoxy)ethan-1-ol;
(iii) 1-(2,5-diaminophenoxy)propan-2-ol;
(iv) 1-(2,5-diaminophenoxy)butan-2-one;
(v) 2-(prop-1-en-2-yloxy)benzene-1,4-diamine; and
(vi) 2-(2-(methylamino)ethoxy)benzene-1,4-diamine, or
a physiologically acceptable salt or solvate thereof.

17. The compound according to claim 16, wherein the compound of formula I is selected from the list:
(i) 2,5-diaminophenoxy)methoxy)methanol;
(ii) 2-(2,5-diaminophenoxy)ethan-1-ol; and
(iii) 1-(2,5-diaminophenoxy)propan-2-ol, or
a physiologically acceptable salt or solvate thereof.

18. A method of dyeing hair or of applying a temporary tattoo, which method comprises applying a composition comprising a compound of formula I or a physiologically acceptable salt or solvate as described in claim 1, or an oxidised derivative thereof.

19. A composition for dyeing hair or tattooing skin, comprising:
a compound of formula I as defined in claim 1; and
water.

20. The composition of claim 19, further comprising a coupling agent.

21. A kit of parts comprising:
(i) a composition according to claim 19; and
(ii) a developing composition comprising an oxidising agent.
